(19) United States
(12) Patent Application Publication
(10) Pub. No.: US 2019/0241943 A1
(43) Pub. Date: Aug. 8, 2019

(54) RAPID PHENOTYPING AND IDENTIFICATION OF MICROBES FROM A COMPLEX MICROBIAL COMMUNITY

(71) Applicant: Duke University, Durham, NC (US)

(72) Inventors: Max Villa, Durham, NC (US); Lawrence David, Durham, NC (US); Rachael Bloom, Durham, NC (US)

(21) Appl. No.: 16/271,564

(22) Filed: Feb. 8, 2019

Related U.S. Application Data
(60) Provisional application No. 62/628,170, filed on Feb. 8, 2018.

(51) Int. Cl. C12Q 1/6851; C12Q 1/686; C12Q 1/689; C12Q 1/06

(52) U.S. Cl. CPC C12Q 1/6851; C12Q 1/06; C12Q 1/689; C12Q 1/686

(57) ABSTRACT

The invention disclosed herein relates generally to the fields of microbiology, ecology and microfluidics. Particularly, the invention disclosed herein provides compositions and methods for isolating, identifying and phenotyping bacteria from complex microbial communities and measuring growth rates of the isolated bacteria in a given environmental condition.

### CROSS-REFERENCE TO RELATED APPLICATIONS

This application claims the benefit of the filing date of U.S. provisional application No. 62/628,170, filed Feb. 8, 2018, the entirety of which is incorporated herein by reference in its entirety.

### BACKGROUND OF THE INVENTION

### Field of the Invention

The invention disclosed herein relates generally to the fields of microbiology, ecology, and microfluidics. Particularly, the invention disclosed herein provides compositions and methods for isolating, identifying and phenotyping bacteria from complex microbial communities and measuring growth rates of the isolated bacteria in a given environmental condition.

### Description of Related Art

Microbial communities are essential for ecosystem function, from their role in human health (e.g., the gut and skin microbiome) to their roles in environment (e.g., soil and water microbiomes). Recognition of the importance of these communities leads to the question of how to connect the structure (i.e., what microbes are present) of these microbial communities to their specific role in the ecosystem. In the case of the human microbiome: identification of specific microbes that are essential for human health or disease prevention.

The current standards in the field (e.g., human microbiome field) include surveys of bacteria and fungi present, measuring which bacteria are present in different places on the body, or during different disease states, and, ultimately, trying to correlate different microbes to different conditions. Metagenomic studies, or whole genome surveys of microbial communities, seek to connect specific bacterial genes to a phenotype. Finally, metabolomic techniques are used to associate specific bacterial products to a phenotype. Combining these techniques together would provide for a better picture of how microbial communities affect and respond to the environment around them.

An important piece that is presently missing is a way to connect bacterial physiology (e.g., the growth) to community structure and function. Growth rate is a fundamental bacterial life history trait. The rate at which a bacterium grows distills many important physiological processes into one variable. Furthermore, the ability to simultaneously measure the growth rates of bacteria isolated from a complex community allows for rapid measurement of the effects of the environment on individual bacterial species. Understanding how individual bacteria of, for example, the gut microbiota respond to diverse environmental factors will enable development of diets or treatments to maintain and promote a healthy gut microbial community. Yet, traditional methods of measuring growth rates for members of the gut microbiota are challenged by the number of unique bacteria inside of one person, and because culturing conditions for most of these bacteria have not yet been described. Problems associated with cultivation hinder traditional methods of measuring growth rates for all the species in the gut. Up to an estimated 80% of microbes from the human gut have not yet been cultivated. Complex culturing conditions, competition in traditional culturing methods, dependence on other bacteria to grow, and low abundances all contribute to the challenge of generating comprehensive culture collections. Any method that comprehensively measures growth rate across the hundreds of species found in the gut microbiota will thus need to address the challenge of isolating uncultivated bacterial taxa.

The complex microbial communities residing inside of our bodies affect human health and disease. Next generation sequencing used to describe community composition has shown abnormal bacterial community profiles associated with disorders such as inflammatory bowel disease, obesity and colorectal cancer. However, composition of bacterial communities based on genotype does not fully predict patient outcomes. For example, metagenomic studies reveal that many microbiota share the same genetic profiles across subjects with different physiological states. The presence of similar genes and pathways suggest an organismal contribution to the host phenotype. Lifestyle and other morphological and biochemical characteristics of individual bacteria are thus needed to understand how the microbiome contributes to human health.

Two crucial ecological traits to characterize are growth rates of individual species and interactions between populations. Growth rate is a fundamental bacterial life history trait, while interactions between bacteria, such as competition or antagonism, are essential for understanding how a microbial community (e.g., the gut microbial community) functions as a complete ecosystem. However, classical approaches for generating growth curves and co-culturing bacteria are inadequate to handle the complexity of a microbiome. For example, in the gut, each human carries several hundred species of bacteria, and unrelated individuals serve as host to unique strains. Given the trillions of possible bacterial species residing in the billions of people in the world, pure culture connections cannot be created for all human-associated bacterial taxa. Each bacterium would require an order of magnitude more variations of conditions (e.g. media and atmosphere) and consumables (such as plates and tubes) to establish co-culture connections and interactions. Therefore, robust tools are needed that reduce the time and resources needed to isolate and study members of the gut microbiota.

### SUMMARY OF THE INVENTION

It is against the above background that this invention provides certain advantages and advancements over the prior art. Specifically, the inventors have found cost-effective and highly efficient high-throughput microfluidics methods for isolating bacteria from complex microbial communities that allow for efficient measurement of growth rates of the isolated bacteria in a given environmental condition.

Although the invention disclosed herein is not limited to specific advantages or functionality, the invention disclosed herein in one aspect provides methods for measuring absolute growth of a bacterial strain from a mixed microbial community, comprising:
(a) isolating a single bacterium from a mixed microbial community by encapsulating the bacterium in an aqueous droplet surrounded by an oil-phase to obtain an encapsulated bacterium;
(b) incubating the encapsulated bacterium under conditions appropriate for growth to obtain a single encapsulated bacterial strain;
(c) extracting DNA from the encapsulated bacterial strain at one or more time points during incubation;
(d) measuring:
   (i) the quantity of total DNA extracted at each time point using quantitative Polymerase Chain Reaction (“qPCR”) via primers that target a variable region within a conserved gene sequence; and
   (ii) the relative abundance at each time point of the encapsulated bacterial strain in the mixed microbial community through sequencing of the same variable region within a conserved gene sequence as in (i) using the same primers as in (i); and
(e) determining absolute growth based on the measurements obtained in (d) at each time point.

Another aspect of the disclosure provides methods for characterizing the microflora in a patient's gut, comprising:
(a) isolating each bacterium within a patient's stool sample by individually encapsulating the bacterium in an aqueous droplet surrounded by an oil-phase to obtain individually encapsulated bacterium;
(b) incubating each encapsulated bacterium under conditions appropriate for growth to obtain encapsulated bacterial strains;
(c) extracting DNA from each encapsulated bacterial strain at one or more time points during incubation;
(d) measuring:
   (i) the quantity of total DNA extracted at each time point using quantitative Polymerase Chain Reaction (“qPCR”) via primers that target a variable region within a conserved gene sequence; and
   (ii) the relative abundance at each time point of the encapsulated bacterial strain in the microflora through sequencing of the same variable region within a conserved gene sequence as in (i) using the same primers as in (i); and
(e) determining absolute growth based on the measurements obtained in (d) at each time point.

In some aspects, the method for characterizing the microflora in a patient's gut further comprises assessing the sensitivity of a bacterial strain from a mixed microbial community to an antibiotic drug, wherein (b) further comprises incubating each encapsulated bacterium both in the presence of an antibiotic drug and in the absence of an antibiotic drug, under conditions appropriate for growth to obtain a single encapsulated bacterial strain; and further comprising (f) measuring the growth of the bacterial strain in the presence of an antibiotic drug and growth in the absence of an antibiotic drug.

In some aspects of the disclosure, the bacterial strain is sensitive to an antibiotic drug when the measurement in (f) demonstrates that the bacterial strain grown in the presence of an antibiotic drug exhibits less than 50% of the growth of the same bacterial strain grown in the absence of an antibiotic drug.

In some aspects, the methods for characterizing the microflora in a patient's gut further comprise assessing the ability of a gut bacterial strain to inactivate a xenobiotic, wherein (b) further comprises incubating each encapsulated bacterium both in the presence of a xenobiotic and in the absence of a xenobiotic, under conditions appropriate for growth to obtain a single encapsulated bacterial strain; and further comprising (f) measuring the growth of the bacterial strain in the presence of the xenobiotic and growth not in the presence of the xenobiotic.

In some aspects, the methods for characterizing the microflora in a patient's gut further comprise assessing the ability of a gut bacterial strain to degrade a prebiotic, wherein (b) further comprises incubating each encapsulated bacterium both in the presence of a prebiotic and in the absence of a prebiotic, under conditions appropriate for growth to obtain a single encapsulated bacterial strain; and further comprising (f) measuring the growth of the bacterial strain in the presence of the prebiotic and growth in the absence of the prebiotic.

Another aspect of the disclosure provides libraries of bacteria that display a targeted phenotype, wherein the bacteria are generated according to the method of the disclosure.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of this invention can be best understood when read in conjunction with the following drawings.

FIG. 1 is images and schematics showing the isolate-to-phenotype pipeline. A. Gut microbial communities are put directly into droplets using a flow-focusing method generating aqueous droplets surrounded by an oil phase. Droplets are then incubated in specified conditions; DNA is extracted, sequenced, and then quantified. B. shows the relative abundance of each bacterial population at each time point (T0, T24 (hours)), which is generated from sequencing of 16S V4 rRNA with Next Gen Sequencing.

FIG. 2 is a graph showing a strong correlation (Spearman rho: 0.7, pvalue=0.04) between measuring absolute growth in droplets (determined by sequencing and qpcr) and absolute growth in a plate reader (determined by OD600).

FIG. 3 is heatmaps of absolute growth in droplets and a plate reader determined by T24 and T0 endpoint measurements. Gut isolates are rows, and antibiotics are columns.

FIG. 4 is a graph showing droplet monocultures grown on different carbon sources. DNA quantity (a.u.) of a single isolate (B. thetaiotamicron, ATCC strain 29148) is a sufficient method to measure the growth of a bacterium in droplets on different substrates.

FIG. 5 is a chart and heatmap showing high throughput measurement of gut bacterial isolate growth on dietary monosaccharides and polysaccharides. End point measurements are shown here on a log 10 scale of bacteria (rows) grown on different dietary carbon sources (columns).

FIG. 6 is a chart and heatmap showing growth response of individual bacteria shown at the genus level (rows) in the presence of glucuronic acid (GluAcid), PNPG, Glucose, and water (H2O) (columns).

FIG. 7 is graphs showing the microbiomes of three individuals (A, B, C) and their differential responses to the presence of the amino acid isoleucine. Shaded bars show the growth of an individual bacteria named at the species level (rows) when the amino acid isoleucine is present. Growth here was determined by an endpoint measurement and compared with the growth of the same bacteria on media without the isoleucine supplement.

FIG. 8 is a chart showing growth rate data from a full fecal community of individual bacteria at the order level, relative to the control growth rate provided in Table 2.

FIG. 9 is an image showing the separate encapsulation of five (5) facultative anaerobic bacteria isolated from the human gut: Escherichia coli, Streptococcus agalactiae, Enterococcus faecalis, Enterobacter cloacae, and Staphylococcus haemolyticus, each of which shows a distinct bacterial morphology within droplets labeled A-E.

FIG. 10 is graphs and images showing concept and validation of droplet growth assay using mock bacterial communities. A. Shows a schematic of bacterial loading and growth in droplets over time. At each time point, droplets are destructively sampled, and B. the 16SrRNA gene is sequenced to establish the relative abundance of each sequence variant. C. qPCR is used on the same samples to determine absolute abundance of the 16S rRNA gene. D. Relative abundance is combined with absolute abundance to form growth curves. An experimental example of MicDrop is depicted in (E-H), featuring an artificial community comprised of B. fragilis, B. longum, E. coli, and E. faecalis gut isolates. This community was loaded via the MicDrop method (E), and then assayed by 16S rRNA composition (F) and qPCR (G) longitudinally using destructive replicate sampling. (H) Fitted growth curves were then inferred.

FIG. 11 is graphs of growth rates across human gut bacterial sequence variants (“SVs”) grown in microfluidic droplets. A. SV abundance estimates and fitted growth curves from a fresh human fecal sample. SVs are shaded by taxonomy. B. Growth rates (p) and C. total growth (number of cells at hour 127−number of cells at hour 0) measured in droplets shaded by phyla. D. Correlations between p and total growth and SV abundance over time in an artificial gut system. E. Total growth in droplets and relative abundance in an artificial gut between days 7 and 14. Dashed axes represent boundaries between high and low abundance in an artificial gut system (vertical line at −3) and high and low growth in droplets (horizontal line at 3).

FIG. 12 depicts a prebiotic utilization screen based on the MicDrop method. A. Shows a schematic of MicDrop prebiotic assay. B. shows results of 96-well plate growth of gut bacterial isolates across 11 carbohydrates. C. provides ROC curve of MicDrop assay results at different growth threshold cut-offs using C as a reference. The black dot indicates the growth threshold that maximizes the true positive rate while minimizing the false positive rate X, depicted in D. E. shows correlation between two different MicDrop sessions (each carried out in triplicate) on the same frozen fecal sample and five different carbohydrates. Points indicate median growth of different SVs across each experimental session.

FIG. 13 shows a graph depicting MicDrop prebiotic assay carried out on fecal samples from nine individuals. A. Microbial carbon preferences for 344 strains from nine healthy human donors (Note this includes growth on glucose alone; 293 strains grew on at least one of the prebiotics). B. The number of primary degraders detected by MicDrop differed by subject (p<0.001, Two-way ANOVA). C. The relative abundance of primary degraders in subject stool also differed by subject and prebiotic (p<0.001, Two-way ANOVA). Subject ordering in B and C are sorted by median.

FIG. 14 is an image showing that microfluidic droplets maintain stability and do not exhibit evidence of coalescing for at least five days. Representative image of microfluidic droplets at hour 127 (5.3 days).

FIG. 15 is a graph showing relative abundance of SVs in the fecal inoculum and inferred SV growth rate in droplets (p>0.9, Spearman correlation).

FIG. 16 is a graph showing a comparison between E. coli grown in plates (measured in CFUs, x-axis) and in microfluidic droplets (measured by qPCR, y-axis; Spearman p=0.95, p=8.7e-9). Cultures of E. coli were grown overnight, then diluted to varying concentrations. These cultures were then simultaneously plated for CFU counting, and DNA was extracted from them for determining cell number via qPCR. These numbers were then compared, and determined that qPCR is comparable method to plate counting as a way to enumerate growing cells.

FIG. 17 is graphs showing sensitivity of inferred parameters to bounds on their values. Each point represents a distinct SV used in our analysis of a human stool sample (See FIG. 11). Parameter bounds in our reported analyses are used along the x-axes; alternative values are shown in results along y-axes.

FIG. 18 is graphs showing threshold determination for droplet prebiotic screen using an artificial community grown both in droplets and well-plates. (A) False positive and true positive rates as a function of SV growth threshold in droplets (where growth is normalized to the maximum growth value for each SV). (B) A threshold of 0.88 maximized Youden's J index, which equally weighs both assay sensitivity and specificity, also referred to as the true positive and true negative rate, respectively.

FIG. 19 is a graph depicting Poisson loading distributions. Tradeoffs exist between the number of droplets loaded with bacteria and how many of those loaded droplets are clonal. MicDrop protocols here balance these tradeoffs by loading droplets at means ranging from 0.1-0.3 (dark gray region).

FIG. 20 is a schematic of droplet production in an anaerobic chamber. Cells are encapsulated in droplets, which are formed by flowing the aqueous bacterial suspension through an immiscible oil via a T-junction on a microfluidic chip (center). Flow is controlled by two syringe pumps (left). Droplet production may be monitored by a microscope equipped with an LCD display (center). After droplets are generated, they are incubated anaerobically (right) until destructive sampling.

### DETAILED DESCRIPTION OF THE INVENTION

All publications, patents and patent applications cited herein are hereby expressly incorporated by reference for all purposes.

Before describing this invention in detail, a number of terms are defined. As used herein, the singular forms “a”, “an”, and “the” include plural referents unless the context clearly dictates otherwise. For example, reference to “an element” means at least one element and can include more than one element.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of this invention.

For the purposes of describing and defining this invention it is noted that the terms “reduced”, “reduction”, “increase”, “increases”, “increased”, “greater”, “higher”, and “lower” are utilized herein to represent comparisons, values, measurements, or other representations to a stated reference or control.

For the purposes of describing and defining this invention it is noted that the term “about” is used to provide flexibility to a numerical range endpoint by providing that a given value may be “slightly above” or “slightly below” the endpoint without affecting the desired result.

For the purposes of describing and defining the invention it is noted that the term “random” when used herein to refer to the method of encapsulation refers to encapsulation that follows the Poisson distribution, as defined herein.

For the purposes of describing and defining this invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

The bacterial communities in, on, and around us impact our health in both positive and negative ways. Each person has their own highly individualized bacterial communities (microbiomes). This disclosure streamlines the process of isolation, identification, and phenotypic testing of individual bacteria from a mixed population. Isolation and identification are both time consuming processes and, combined with phenotyping (how well a bacteria responds to an antibiotic treatment, what carbon source a bacteria is able to use, or any microbial responses to an ingested drug, etc.), are all necessary for treatment of harmful pathogens, the promotion of beneficial bacteria, and the efficacy of a drug for a patient. Streamlining this process is imperative as individuals' microbiomes (the bacterial communities in and around them) can be as distinct as a fingerprint; in addition to bacterial communities from different people being distinct, similar bacteria from different people behave differently.

Microfluidics has made impressive progress isolating uncultivated bacteria. Recent attempts to cultivate bacteria from complex environments have relied on microfluidic devices that scale down the amount of reagents needed to culture and scale up the number of organisms able to be isolated at one time. These microfluidic devices use complex diffusion chambers, in the case of the iChip, which captures bacteria in chambers then exposes them to complex environmental conditions. The iChip is thus able to identify novel species that cannot grow in current laboratory media. Similarly, another device, the SlipChip, confines individual bacteria into microscopic wells on a chip, and the colonies trapped in these wells are able to be propagated for isolation. Both of these microfluidic devices were specifically designed for isolation of bacteria, but neither is well-equipped to perform high-throughput phenotypic characterization of bacterial isolates.

The diversity of the gut microbiota and other complex microbial communities is maintained in part by interactions between bacteria. Antagonism between populations is one type of interaction that contributes to the diversity, and these interactions are often mediated by small molecules including antibiotic compounds. Recently, there has been renewed interest in mining the gut microbiota for yet-undiscovered molecules that could have known antimicrobial activity. Among the uncharacterized portion of the gut microbiota there may be useful treatments to prevent the colonization of pathogens. To identify interactions between two species of bacteria, microbiologists traditionally co-culture bacteria, grow them together, and look for inhibition. For example, to look at the interactions between members of the genera Vibrio, over 35,000 possible interactions were screened to understand the population structure of microbes in the ocean. Similarly, over 30,000 isolates from the gut microbiota were screened for antimicrobial activity against the pathogen Clostridium difficile and a novel two-component antimicrobial peptide was identified. Other attempts to identify probiotic candidates have included computationally predicting the presence of over 3,000 small-molecule biosynthetic gene clusters in the human gut microbiome. The presence, structure, and antibacterial activity was subsequently verified for one of the compounds. Culturing two or more bacterial species together is the primary way of identifying interactions between species. An individual gut microbiota contains as many as 1,000 different species; therefore, to test all possible pairwise interactions (499,500) would be burdensome, requiring over 5,000 96-well plates for culturing alone.

There is thus a need in the art to develop approaches that both isolate bacteria and allow high-throughput phenotypic characterization, as well as high-throughput identification of antagonistic and other interactions between bacterial members of a microbial community.

In general, the disclosed materials, methods, and apparatus provide cost-effective and highly efficient improvements in rapid methods for isolating bacteria from complex microbial communities. Specifically, the inventors have founds that use of the high-throughput microfluidic methods of the disclosure allows for efficient measurement of growth rates of the isolated bacteria in a given environmental condition.

Measuring Growth Rates of Bacterial Species within Complex Microbial Communities

The human gut microbiome is a complex ecosystem affecting human health and disease. Due to the amount and variation of the bacterial populations that reside inside our gastrointestinal tract (e.g., the gut microbiota), however, ecological traits of individual bacteria remain uncharacterized. Two traits essential to understanding the functioning microbial community are: growth rates of individual bacterial populations, and corresponding interactions among bacterial populations based on characteristics of individual bacterial strains. Use of traditional methods to isolate, culture and assess phenotypic characteristics of individual bacterial strains from a mixed microbial community, such as the gut, would be impractical.

The present disclosure provides, in part, high throughput methods to measure growth of individual bacterial strains from mixed microbial communities, and methods to assess properties such as antibiotic susceptibility profiles, drug breakdown on the human gut, and response of bacterial communities to the addition of foreign compounds, for hundreds of species in a single day. Thus, one aspect of the present disclosure provides measuring isolated growth in droplets without the need for double-emulsion techniques or droplet sorting. Instead, the present disclosure combines single-emulsion (water-in-oil) microfluidic droplet protocols with hi throughput DNA sequencing techniques (such as, for example, qPCR and 16S rRNA sequencing) to isolate and assay individual members of a microbiota in picoliter droplets (“MicDrop”).

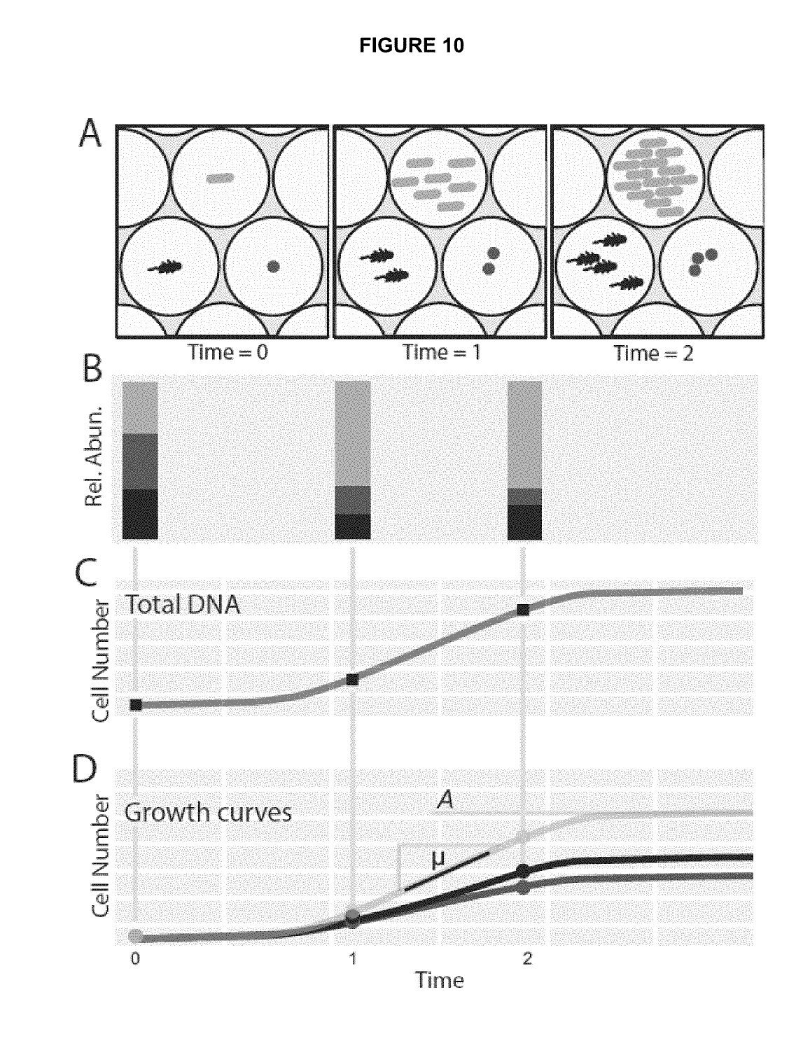

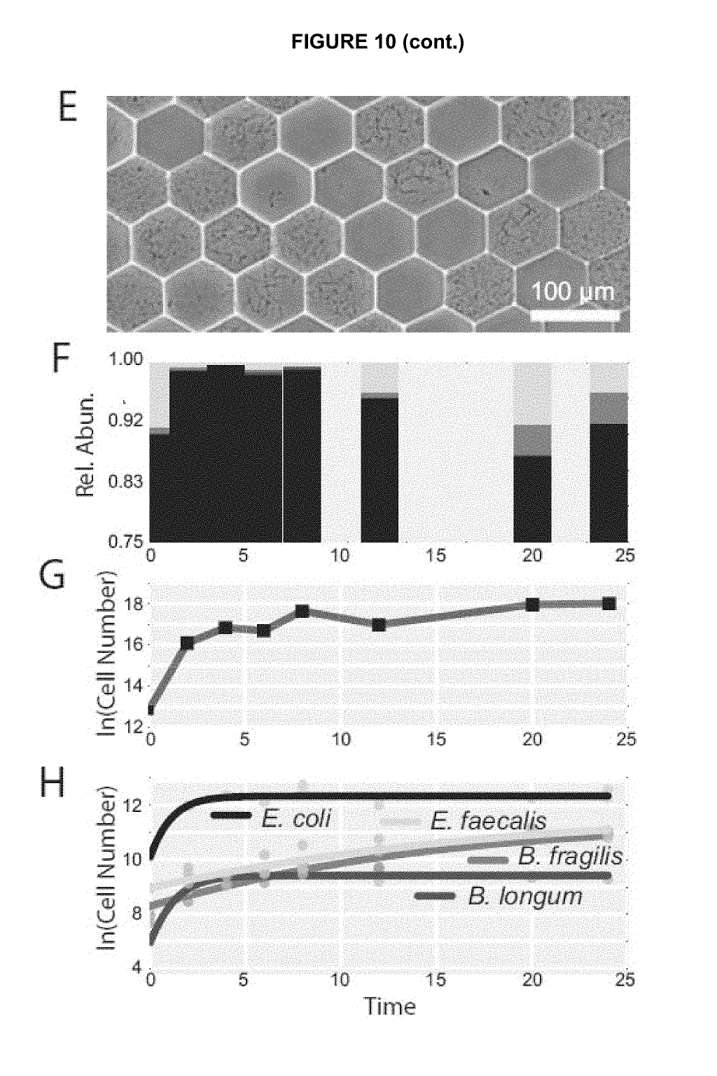

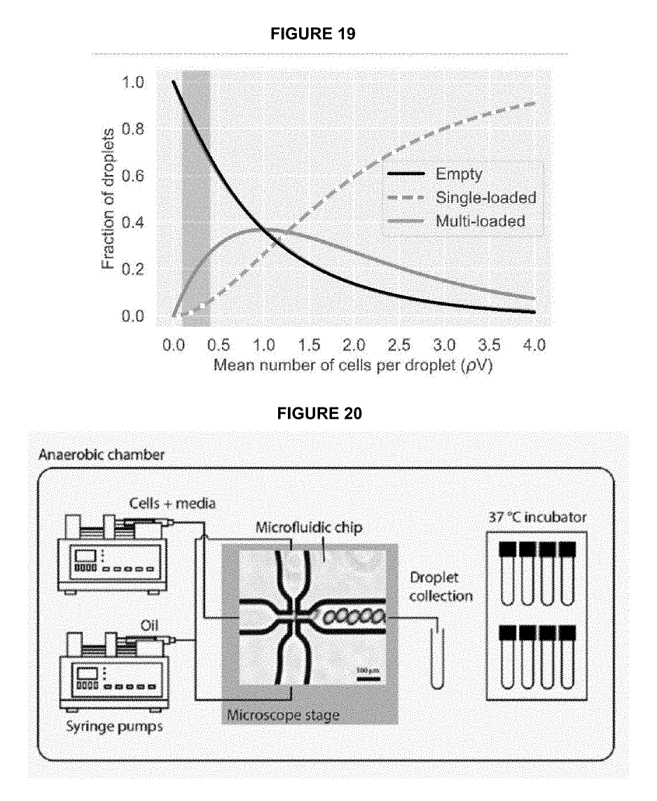

Bacterial cells from a mixed microbial community are individually isolated and encapsulated in aqueous droplets surrounded by an oil phase using the Poisson distribution. (FIG. 10, FIG. 19). Each encapsulated bacterium is incubated under conditions appropriate for growth to obtain a single encapsulated bacterial strain; DNA is then extracted from the encapsulated bacterial strain at one or more time points during incubation. The extracted DNA is then used to measure total DNA at each time point using molecular techniques, and relative abundance at each time point using DNA sequencing, and determining absolute growth from these measurements.

Using MicDrop, growth curves can be generated for dozens of distinct SVs in a single experiment, which in turn allow for prediction of long-term microbiota dynamics of an artificial human gut. In some aspects, the present disclosure provides that the isolated encapsulated droplets may be cultured to characterize dietary polysaccharide degradation in mixed bacterial communities, such as gut bacteria. Together, these findings showcase the potential for microfluidic droplet techniques to characterize the growth and function of individual bacterial strains from complex gut microbial communities using the disclosed high-throughput methods.

In some aspects, the present disclosure provides a method to measure the growth rate of bacteria from mixed communities in a high-throughput manner. One of skill in the art recognizes that the standard method for growth rate is an essential variable to measure because it encompasses so many aspects of bacterial physiology. The ability to measure growth rate of bacteria from a community provides a better picture of an individual microbe's role in the larger community. High-throughput droplet microfluidic techniques are used herein to measure the growth of individual members of the gut microbiota, identify isolated bacterial strains which are resistant or susceptible to antibiotic drugs, and/or which are able to grow on prebiotic compounds. Thus, one aspect of the present disclosure provides droplet microfluidic techniques to isolate an individual bacterium from a complex environment (i.e., as one non-limiting example, from the human gut microbiota). In some embodiments of the present disclosure, once the individual bacteria are isolated, next generation DNA sequencing is combined with quantifying the DNA to measure the growth rate of bacterial populations isolated within the droplets. In some embodiments, the DNA may be quantified by, as one non-limiting example, quantitative Polymerase Chain Reaction (qPCR). These novel techniques make previously uncultivated members of the gut microbiota amenable for experimentation and increase understanding of how individual members of the gut microbiota grow, interact, and contribute the gut microbial community and, subsequently, to the health of the host.

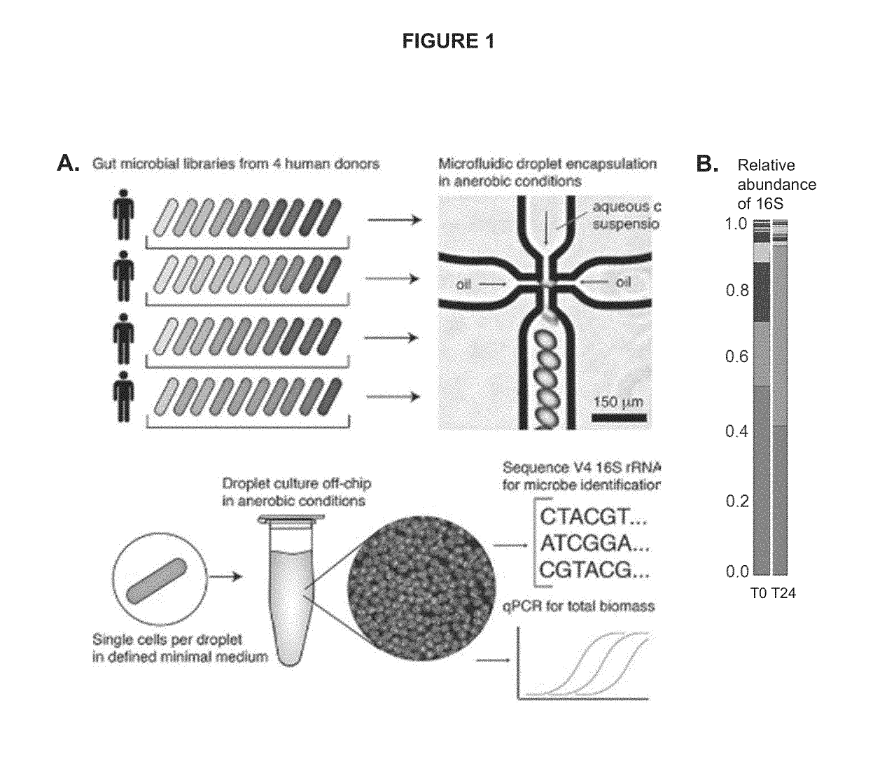

In some aspects, the present disclosure provides an improved method for rapid and accurate determination of growth rates without the need for generating a full growth curve. In some embodiments, the present disclosure provides methods of determining growth rates of bacteria within a complex community based on only one or two culture time points. The improved method for rapidly isolating and measuring the growth rate of members of a bacterial environmental community comprising, consisting of, or consisting essentially of encapsulating individual bacteria in droplets to produce aqueous droplets of a chosen growth media surrounded by an oil-phase; culturing the anaerobic bacteria in an anaerobic chamber; incubating the encapsulated droplets at an appropriate temperature; extracting DNA from the droplets; measuring both relative abundances of different taxa and the amount of DNA present using primers (such as the 16S V4 primers); and computing the absolute growth using one or more culture time points. (FIG. 1).

In some embodiments of the disclosure, isolation and encapsulation of a bacterial strain is random. In some embodiments, this technique generates about 10,000 to about 100,000 droplets per minute, for example, about 10,000 to about 80,000 droplets per minute, or about 10,000 to about 60,000 droplets per minute, or about 30,000 to about 80,000 droplets per minute, or about 30,000 to about 70,000 droplets per minute, or about 30,000 to about 60,000 droplets per minute, or about 30,000 to about 50,000 droplets per minute, or about 40,000 to about 80,000 droplets per minute, or about 40,000 to about 70,000 droplets per minute, or about 40,000 to about 60,000 droplets per minute, or about 45,000 to about 55,000 droplets per minute, or about 48,000 to about 52,000 droplets per minute, or about 40,000 droplets per minute, about 50,000 droplets per minute, or about 55,000 droplets per minute.

The generated droplets capture individual bacteria within the droplets. The size and scale of the droplets makes individually encapsulating a single bacterium feasible. Thus, in some embodiments, the individual droplets may average from about 10 μm to about 1 mm in diameter, for example, about 10 μm to about 750 μm, or about 10 μm to about 500 μm, or about 10 μm to about 250 μm, or about 10 μm to about 200 μm, or about 10 μm to about 150 μm, or about 10 μm to about 100 μm, or about 50 μm to about 1 mm, or about 50 μm to about 750 μm, or about 50 μm to about 500 μm, or about 50 μm to about 250 μm, or about 50 μm to about 200 μm, or about 50 μm to about 150 μm, or about 50 μm to about 100 μm, or about 100 μm to about 1 mm, or about 100 μm to about 750 μm, or about 100 μm to about 500 μm, or about 100 μm to about 250 μm, or about 100 μm to about 200 μm, or about 75 μm to about 125 μm, or about 80 μm to about 120 μm, or about 90 μm to about 110 μm, or even about 100 μM in diameter.

Random encapsulation of bacteria in a droplet follows the Poisson distribution:

$$P ( n , \overline{n} ) = \frac{{\overline{n}}^{n} {e}^{- \overline{n}}}{n !} ,$$

where n is the droplet occupancy (i.e. 0, 1, . . . cells/droplet) and n̅ is the average number of cells per droplet given by: n̅=ρV, where V is droplet volume and p is cell density. Thus, when about 10% of droplets have bacteria in them, over about 90% of those droplets have clonal populations of bacteria. The large amount of empty droplets is rapidly overcome by the total number of droplets produced. In some embodiments of this disclosure, a bacterium is individually encapsulated in a droplet, using a high-throughput method. Droplets may be generated by any method known in the art, such as, without limitation, a droplet chip, centrifugation, mechanical shaking of two immiscible liquids (such as water and oil), microfluidic chip, etc. In one embodiment, droplets are generated using a 6-junction droplet chip available from DOLOMITE® microfluidics. The droplet chip produces aqueous droplets of a chosen growth media surrounded by an oil-phase (i.e., water-in-oil droplets). Any suitable hydrophobic material having suitable viscosity and/or interfacial tension may be used for the oil phase. Hydrophobic materials suitable for use in the methods of the disclosure include, but are not limited to, mineral oil, silicon oil, perfluorinated oil, and fluoro-carbon oil (such as one available from BIORAD®.)

In some embodiments, the entire microfluidic setup is placed within an anaerobic (e.g., oxygen-free) chamber to culture the bacteria. In some embodiments, the bacteria are encapsulated within and incubated in an aqueous droplet containing nutrient-rich culture media. One of skill in the art would immediately recognize that this encompasses any suitable kind of media classified as nutrient-rich, including, but not limited to: Brain Heart Infused (BHI) medium, Gifu Anaerobic Medium (GAM), and modified Gifu Anaerobic Medium (mGAM). In some embodiments, the bacteria are encapsulated within, as well as incubated in, an aqueous droplet containing defined media. One of skill in the art would immediately recognize that this encompasses any suitable media classified as defined media, including media with known and defined sources of carbon, nitrogen, and salt (NaCl) which is necessary for growth of certain type of bacteria. In some embodiments, the encapsulated bacteria are grown on defined media containing one or more of the following non-limiting carbon sources: mannose, arabinose, fructose, glucose, xylan, lamanarin, pullalan, levan, rice-starch, arabinogalactan, inulin, fructooligosaccharide (FOS), galactose, glucuronic acid, pectin, galactomannan, guar gum, chitin, galacto-oligosaccarides, cellobiose, dextran, or beta-glucan. In some embodiments, the encapsulated bacteria are grown on defined media containing one or more amino acids or amino acid derivatives as a nitrogen source: Glycine (Gly), Alanine (Ala), Valine (Val), Leucine (Leu), Isoleucine (Ile), Proline (Pro), Phenylalanine (Phe), Tyrosine (Try), Tryptophan (Trp), Serine (Ser), Threonine (Thr), Cysteine (Cys), Methionine (Met), Asparagine (Asn), Glutamine (Gin), Lysine (Lys), Arginine (Arg), Histidine (His), Aspartate (Asp), Glutamate (Glu), Selenocysteine (Sec), or derivatives thereof. In some embodiments, the encapsulated bacteria are grown under a range of conditions, including changes to oxygen (O₂), pH, temperature, and salt content.

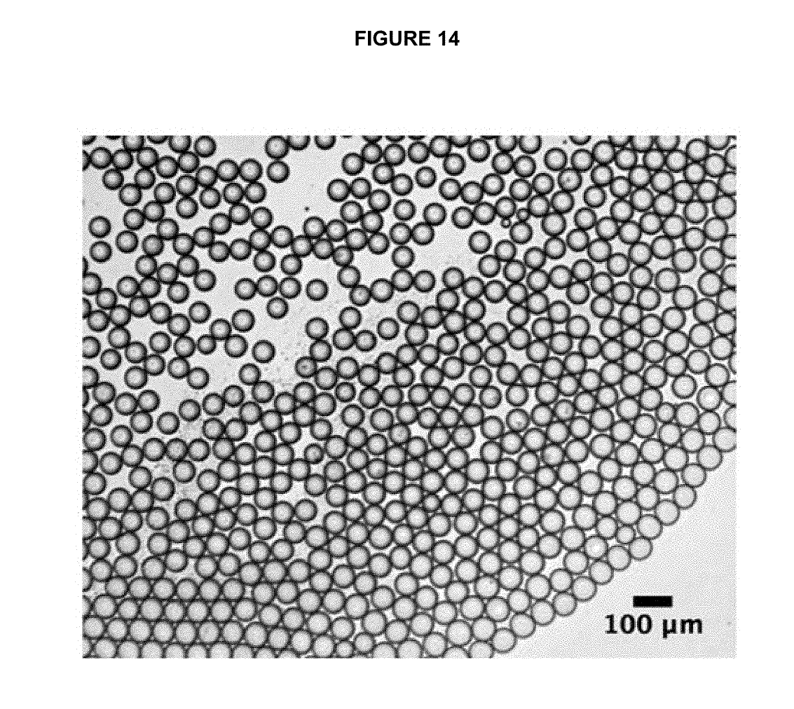

In some aspects, growth rates of the bacteria isolated in droplets are characterized through DNA sequencing combined with quantification of the DNA. DNA sequencing can be performed by any known method, including but not limited to next generation or high-throughput DNA sequencing. Similarly, the DNA can be quantified by any known method, including but not limited to, by quantitative PCR (qPCR). In some embodiments, once bacteria are encapsulated, the droplets are incubated at an appropriate temperature (e.g., 37° C.). In some embodiments, DNA is extracted from the incubating droplets at set time points (e.g., at 60 minutes, or at 6 hours, or at 12 hours, or at 22 hours, or at 24 hours, or at 36 hours (T36), or at 48 hours (T48), or at 60 hours (T60), or at 72 hours (T72), or at 84 hours (T84), or at 96 hours (T96), or at 108 hours (T108) or at 120 hours (T120) or later). In some embodiments, DNA is extracted from the incubating droplets at one or two set time points (e.g., at 0 minutes (T0), or at 12 hours (T12), or at 24 hours (T24), or at 36 hours (T36), or at 48 hours (T48), or at 60 hours (T60), or at 72 hours (T72), or at 84 hours (T84), or at 96 hours (T96), or at 108 hours (T108) or at 120 hours (T120) or later). Quantitative growth measurements using qPCR of the 16S rRNA gene sampled every two hours from liquid cultures showed similarity to E. coli grown on plates (P=0.95, p=8.7e-9; Spearman correlation; FIG. 16). Droplet stability experiments suggest that isolate growth can be measured for up to five (5) days. (FIG. 14.)

In some aspects, the present disclosure provides that two different measurements are performed on the extracted DNA. In some embodiments, both relative abundances of different taxa and the amount of DNA present are measured using suitable primers. In some embodiments, the primers include the 16s rRNA primers. In some embodiments, the primers include the 16s V4 rRNA primers: forward primer GTGCC AGCMG CCGCG GTAA (SEQ ID NO:1) and reverse primer GGACT ACHVG GGTVVT CTAAT (SEQ ID NO:2), wherein the primers target a variable region within conserved gene sequence 16s ribosomal RNA (“rRNA”).

In some aspects, the present disclosure provides that growth is determined by multiplying the total amount of DNA from quantitative qPCR data and the relative abundances from sequencing, and used to create absolute growth measurements. In some embodiments, absolute growth measurements are determined from one or two culture time points. In some embodiments, absolute growth measurements are determined from end point measurements, such as by non-limiting example, after zero minutes (T0) or immediately after droplet making, or after incubation of 60 minutes, or 6 hours (T6), or 12 hours (T12), or 24 hours (T24), or at 36 hours (T36), or at 48 hours (T48), or at 60 hours (T60), or at 72 hours (T72), or at 84 hours (T84), or at 96 hours (T96), or at 108 hours (T108) or at 120 hours (T120) or later. In this way the growth rates of individual bacteria can be measured directly from an environmental community in a specific condition.

### Determining Absolute Growth in Droplets Using Endpoint Measurements

In some aspects, the present disclosure provides that DNA sequencing (including by, for example, next generation or high-throughput DNA sequencing) combined with measuring the DNA (including by, for example, quantitative PCR (qPCR)) allows for characterization of absolute numbers of bacteria isolated in droplets. In some embodiments, once encapsulated, the bacterial droplet is cultured in appropriate medium and incubated at an appropriate temperature. In some embodiments, this disclosure provides for sampling of the droplets at a set time point, which may be immediately after generation of the droplets (time zero, or T0), and/or a certain time point after incubation of the droplets, such as, by non-limiting example, 12 hours after incubation (T12) or 24 hours after incubation (T24), or 36 hours after incubation (T36), or 48 hours after incubation (T48), or 60 hours after incubation (T60), or 72 hours after incubation (T72), or 84 hours after incubation (T84), or 96 hours after incubation (T96), or 108 hours after incubation (T108) or 120 hours after incubation (T120), or later. DNA is extracted from the droplets and, in some embodiments, both relative abundance of different taxa and the amount of DNA present are measured using the 16S V4 rRNA primers. See, e.g., FIG. 1B. In some embodiments, to compute absolute growth of an individual bacterium, the measured DNA value (such as, for example, by quantitative qPCR) is multiplied by the relative abundances from DNA sequencing. In this way, it is possible to determine the absolute growth of an individual bacterium directly from an environmental community (such as a fecal sample) under a specific condition.

### Effect of Culture Conditions on Bacterial Growth in Droplets

Bacterial growth on a defined medium is an important tool for phenotyping. By using a defined media, a function of an isolated bacterium can be determined. Use of the disclosed methods provides for rapid phenotyping of bacteria from a complex community, such as the gut. In some embodiments within this disclosure, this function is growth or absence of growth on defined medium with the addition of a substrate of interest. In some embodiments disclosed herein, the substrate of interest is a known carbon source, and includes carbon sources of varying complexity. In some embodiments disclosed herein, the substrate of interest is a known nitrogen source, such as, for example, an amino acid or a derivative thereof. In some embodiments disclosed herein, the substrate of interest is pH level. In some embodiments disclosed herein, the substrate of interest is oxygen concentration. In some embodiments disclosed herein, the substrate of interest is concentration of salt (NaCl). In some embodiments disclosed herein, the substrate is an antibiotic drug. In certain embodiments, the antibiotic drug can be, for example, amoxicillin, amoxicillin clavulanate, ciprofloxacin, gentamicin, kanamycin or ampicillin. In some embodiments disclosed herein, the substrate is a xenobiotic.

After incubation of the encapsulated droplets, in certain aspects of this disclosure, endpoint growth can be determined by absolute growth measured by DNA extraction, sequencing and qPCR. In certain aspects of this disclosure, the absolute growth of an individual bacterium in a defined medium with the addition of one carbon source can be determined and compared to the absolute growth measurement of the same bacteria in the defined medium with only the addition of water, as a control. Thus, in certain embodiments of this aspect, this comparison allows for assessing the ability of an individual microbe to use a single carbon source for growth. In the same way, in certain embodiments disclosed here, comparison of growth of an encapsulated bacterium grown in defined medium with the addition of any individual substrate, with growth of the same encapsulated bacterium grown in defined medium with the addition of water as a control is able to assess the ability of that individual microbe to use the corresponding substrate for growth.

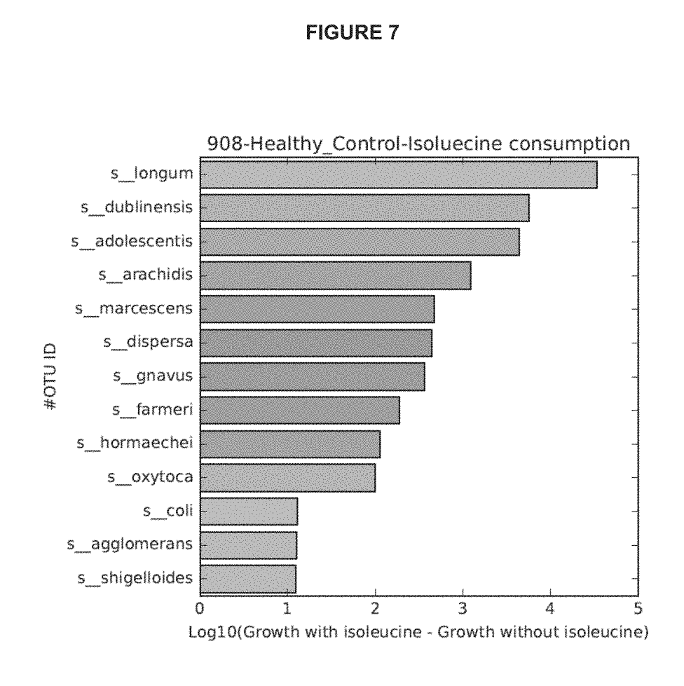

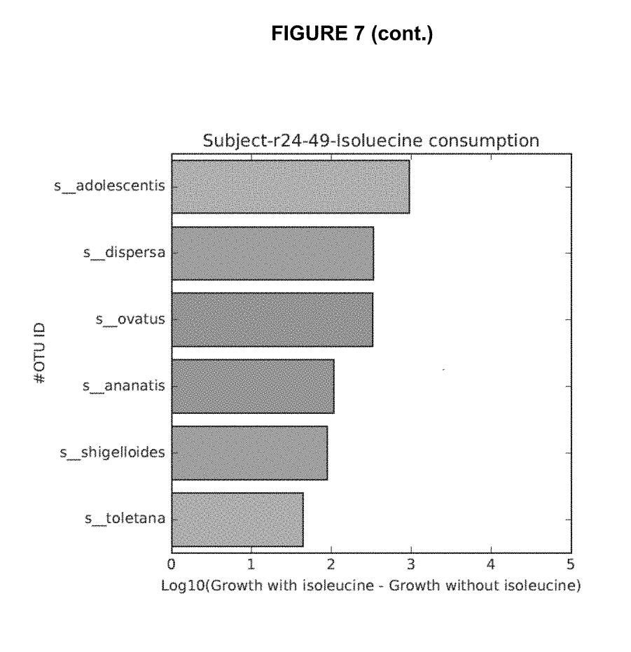

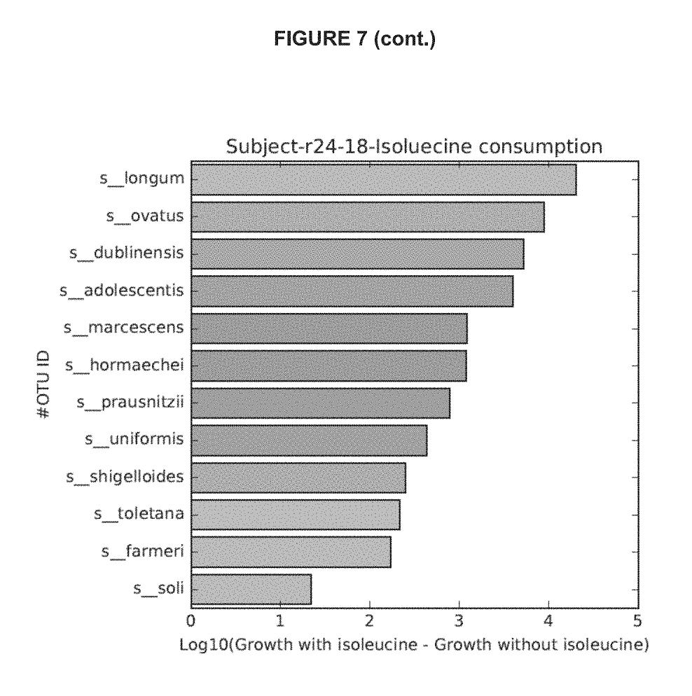

Use of the methods disclosed herein not only allows for rapid phenotyping of bacteria within the gut microbiota of one individual, but also allows for rapid phenotyping of the same bacterial species between individuals, and how the same bacterial species from different individuals can grow differently on a given substrate when added to defined medium. For example, the role that microbiota play in amino acid metabolism is thought to play a role in human metabolic syndrome. Using defined medium, the growth of bacteria in the presence of limited nutrient conditions, such as amino acid supplementation, can be examined. In some embodiments disclosed herein, the growth of encapsulated bacteria cultured in defined medium with and without the addition of an amino acid is compared. Bacteria that increase in growth when the amino acid is present are consumers of the amino acid. In some embodiments disclosed herein, putative amino acid-consuming bacteria can be compared between healthy individuals and individuals with a metabolic syndrome. (FIG. 7). In the same way, in some embodiments disclosed herein, a comparison of growth of an encapsulated bacterium grown in defined medium with the addition of any individual substrate, with growth of the same encapsulated bacterium grown in defined medium with the addition of water as a control would allow for assessing the ability of that individual microbe to use the corresponding substrate for growth and determine how each bacterial species differs in function between different individuals. Hence, the disclosed method can be used as an efficient tool to compare the function of microbial communities between individuals.

### Determining Antibiotic Susceptibility in Droplets Using Endpoint Measurements

Antibiotic testing is an important clinical microbiology practice, where clinicians commonly test pathogens for antibiotic sensitivity to administer effective antibiotics and reduce the overuse of antibiotics. The common practice is to culture an isolated microbe and perform zone of inhibition tests with antibiotic discs. This involves a time consuming multistep process of isolating and identifying the targeted pathogen, culturing the targeted pathogen, and performing a multiday antibiotic susceptibility test. Use of the droplet method disclosed herein provides a streamlined process, not only for isolating and identifying individual bacteria, but also for providing one pipeline to more efficiently and cost-effectively isolate and identify individual bacteria, as well as test for resistance and susceptibility to antibiotics.

Assays for determining antibiotic resistance and susceptibility of a bacterium in droplets are disclosed herein. Bacteria from a mixed community are isolated and encapsulated in droplets in media with an antibiotic drug and without (control). The isolated encapsulated bacteria are incubated in the droplets under appropriate conditions, and DNA is sampled at one or more set time points during incubation of the encapsulated droplets. Absolute growth is then computed as previously disclosed herein. A bacteria is sensitive to the antibiotic if the bacteria grew less than 50% of its growth without the antibiotic (control).the gut are isolated and cultured by suitable standard methods. In certain embodiments, the encapsulated droplets are cultured under appropriate conditions for 60 minutes, or 8 hours, or 12 hours or 24 hours, or more. In some embodiments, the antibiotic drug can be, for example, amoxicillin, amoxicillin clavulanate, ciprofloxacin, gentamicin, kanamycin or ampicillin. In some embodiments, growth of the bacteria in droplets is further compared to growth of isolated bacteria by standard culture, wherein the cultured isolated bacteria from the gut are simultaneously put in a plate reader in the presence of antibiotics. In some embodiments, after sequencing the DNA from one or more set time points during incubation of the encapsulated droplets, absolute growth is computed by taking the absolute number of cells at the endpoint of incubation (e.g., 24 hours (T24)) and dividing that by the absolute number of cells at the point the droplets are generated (T0).

This assay requires isolated bacteria to grow in droplets in the control condition because of the inability to determine whether failure of an isolated bacteria to grow in droplets under the control conditions is attributed to sensitivity to an antibiotic or inability to grow in the droplet environment. There may be multiple reasons why a bacterial isolate does not grow in droplets, for example suitability of media or a lack of a solid substrate. After analyzing the bacteria that were able to grow in droplets, it was determined that bacteria that grow less than 50% of its growth under the control indicates sensitivity to the antibiotic. Using this criteria, and the growth in the plate reader as the true value for antibiotic susceptibility, the disclosed assay correctly predicted antibiotic sensitivity of 21 out of 28 combinations (accuracy of 75%).

### Growth Response to Xenobiotics

Xenobiotics are a catch-all term used to describe ingested chemicals that are foreign (or not naturally produced) by an organism. In the context of the gut microbiome, the term commonly refers to drugs, including antibiotics. Other xenobiotics may include industrial chemicals, food additives, and pollutants. Without metabolism by the body, many xenobiotics would reach toxic concentrations. In their gastrointestinal tract, humans have various mechanisms in place to inactivate xenobiotics, many of which include the microbiome. Gut microbes are able to alter the toxicities and bioavailability of many drugs, which has relevance to dosing and drug development. Microbiome response to xenobiotics therefore is a relevant to microbiome studies and drug metabolism.

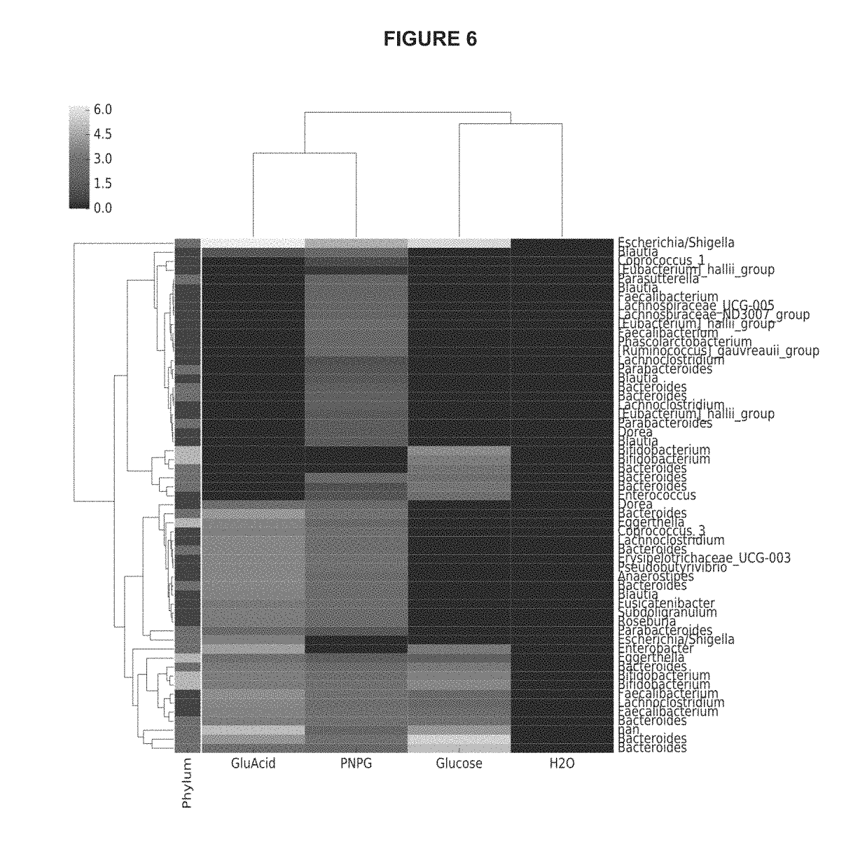

Thus, in certain aspects of this disclosure, the methods of the invention can be used to identify or screen bacteria from the gut that can utilize, and thus inactivate, specific xenobiotic compounds. In certain embodiments of this aspect, the methods of the invention can be used to screen gut bacteria for beta-glucuronidases, which are present in bacteria that can inactivate certain man-made therapeutics. In certain embodiments of this aspect, the screened bacteria exhibited growth on the monosaccharides glucose and glucuronic acid, as well as a conjugated form of glucuronic acid: 4-Nitrophenyl-Beta-D-glucuronic acid (PNPG). (FIG. 6). Bacterial growth on PNPG requires beta-glucuronidase (4-Nitrophenyl-Beta-D-glucuronic acid) to liberate glucuronic acid bound to the pheol group of PNPG. Thus, in some embodiments, the present disclosure provides for identification of bacterial taxa that contain putative beta-glucuronidases. Using the method disclosed herein, bacteria are isolated by encapsulation in droplets of defined media with or without a specific xenobiotic. DNA is sampled from the encapsulated droplets at one or more set time points during incubation, and absolute growth is then computed as previously disclosed herein. Bacterial strains which exhibit growth by utilizing a specific xenobiotic compound may be able to inactivate that xenobiotic compound, and are identified as putative beta-glucuronidases.

### Prebiotic Consumption by Human Gut Microbes

Assays to determine bacterial utilization of prebiotics, or carbohydrates that are indigestible by humans but can stimulate the growth of gut microbes are known in the art. In carbon screens, bacteria are cultured in defined media containing a carbohydrate as the sole carbon source. Microbes that grow are assumed to be capable of utilizing the carbohydrate and are termed “primary degraders”. Bacterial prebiotic metabolism is incompletely understood, particularly with regards to the origins of wide inter-individual variation in microbiota metabolic potential. However, prebiotic utilization assays are an attractive application of MicDrop because they can be reduced to binary outcomes of “growth” or “no growth”, without requiring prolonged droplet sampling. The biology of primary degraders is also of increasing interest because bacterial metabolism of prebiotics leads to the growth and activity of gut microbes with multiple beneficial impacts on host health. Thus, in certain embodiments, the methods of the invention can be used to identify bacteria from the gut that can utilize specific prebiotic compounds for growth. Prebiotic compounds include carbohydrates which are not digestible by humans, but in which specific gut microbes can grow. Using the method disclosed herein, bacteria are isolated by encapsulation in droplets of defined media with or without a prebiotic carbohydrate. DNA is sampled from the encapsulated droplets at one or more set time points during incubation, and absolute growth is then computed as previously disclosed herein. Bacterial strains which exhibit growth by utilizing a specific prebiotic compound are primary degraders of that prebiotic compound.

The invention is further described in the following examples, which do not limit the scope of the invention described in the claims.

### EXAMPLES

### Example 1: MicDrop Procedure

(A) Droplets were made on a microfluidic chip (6-junction droplet chip, Dolomite Microfluidics). Bacterial media varied by assay; for the oil phase, fluorinated oil and surfactant mixture 1% Picosurf (SPHERE FLUIDICS®) in Novec 7500 (3M®) was used. One day prior to performing the droplet assay, all reagents including carrier oil, culture media, and carbon solutions were equilibrated to the anaerobic atmosphere in an anaerobic chamber (COY®). The fecal inoculum optical density at 600 nm was recorded and diluted according to the Poisson distribution:

$$P ( n , \overline{n} ) = \frac{{\overline{n}}^{n} {e}^{- \overline{n}}}{n !} ,$$

where n is the droplet occupancy (i.e. 0, 1, 2, . . . cells/droplet) and n̅, is the average number of cells per droplet given by: n̅=ρV, where V is droplet volume and ρ is cell density. Assays were performed at a n̅ of 0.1-0.3 to minimize the number of droplets loaded with more than one cell. Thus, for a fixed droplet volume and n̅, the target cell concentration can be obtained from:

$$ρ = K \frac{\overline{n}}{V} ,$$

where K is a constant to convert CFUs/mL to OD₆₀₀ determined from replicate CFU assays. Syringe pumps were used to control the flow rates of the oil and cell suspension (NE-1000 Single Syringe Pump, New Era Pump Systems®). Following the culture period, droplets were loaded into chambered slides (C10283, INVITROGEN®) or directly onto glass slides and observed with Phase and/or Darkfield microscopy (NIKON®) to examine growth and the appropriate loading. All steps of cell encapsulation and culture were performed in an anaerobic chamber.

### Collection and Preparation of Fecal Inoculum for Artificial Gut and MicDrop Growth Dynamic Assays

(B) Stool was collected from human donors under a protocol approved by the Duke Health Institutional Review Board (Duke Health IRB Pro00049498). Inclusion criteria limited this study to healthy subjects who could provide fecal samples at no risk to themselves, had no acute enteric illness (e.g. diarrhea) and had not taken antibiotics in the past month. Fresh stool samples were collected in a disposable commode specimen container (FISHER SCIENTIFIC®, Hampton N.H.). Intact stool was moved within roughly 15 minutes of bowel movement into anaerobic conditions. The sample was prepared for inoculation in an anaerobic chamber (COY®). A 5 g stool aliquot was weighed into a 7 oz filtration bag (Nasco WHIRL-PAK®) and combined with 50 mL of mGAM media (modified Gifu Anaerobic Media, HIMEDIA®) that was pre-reduced overnight in an anaerobic chamber. The mixture was homogenized in a stomacher (SEWARD® Stomacher 80) on normal speed for 1 minute under atmospheric conditions to make a total of 100 mL of inoculum. The supernatant was decanted into beakers and loaded into syringes for inoculation into the artificial gut or filtered through a 50 μm filter (CELLTRICS®) and diluted and loaded into droplets.

### Droplet DNA Extraction, PCR Amplification, and DNA Sequencing

(C) To extract DNA from droplets, excess oil was removed by pipetting and water-in-oil emulsions were broken by adding an equal amount of 1H,1H,2H,2H-Perfluoro-1-octanol (PFO, VWR®) and briefly vortexed. Then, the samples were briefly centrifuged (<200 g) to separate the aqueous and oil phases by density. The aqueous solution was transferred to a new tube, and DNA was extracted using an kit (QIAGEN® #12224). DNA was extracted from artificial gut and stool samples using a 96-well PowerSoil kit (QIAGEN® #12888). For all samples, the V4 region of the 16S rRNA gene was barcoded and amplified from extracted DNA using with custom barcoded primers, using published protocols. 16S rRNA amplicon sequencing was performed on an Illumina MiniSeq with paired-end 150 bp reads. Only samples with more than 5,000 reads were analyzed, to remove outlying samples that may have been subject to library preparation or sequencing artifacts. The 16S rRNA nucleotide sequences generated in this study will be made available at the European Nucleotide Archive under study accession number TBD. Total bacterial abundances from droplet cultures were estimated by qPCR for bacterial 16S rRNA using the same primers used in the DNA sequencing protocol. Amplification during the qPCR process was measured with a Real-Time PCR system (CFX96 Real-Time System, BIORAD®) using E. coli DNA at a known cell concentration as a reference.

### Example 2: Isolation of Bacteria in Individual Droplets and Determining Absolute Growth Using Endpoint Measurements

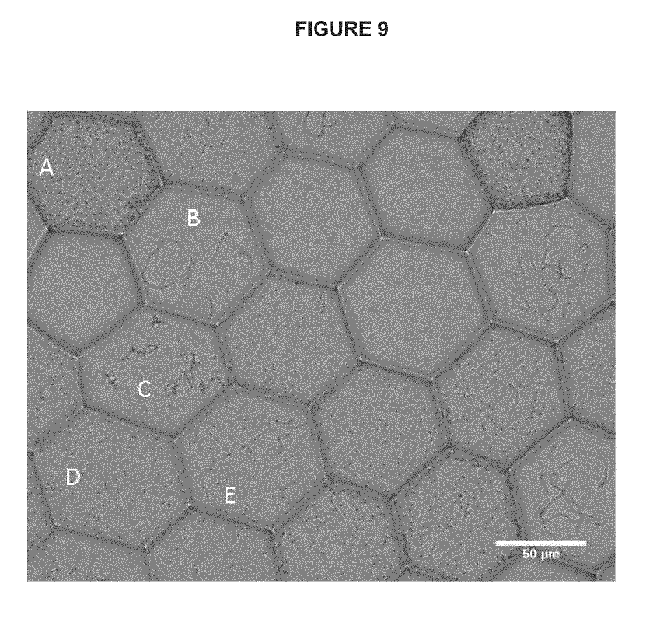

(A) Human stool samples were obtained and used to isolate individual bacterium within the human gut: Escherichia coli, Streptococcus agalactiae, Enterococcus faecalis, Enterobacter cloacae, Staphylococcus haemolyticus. These five facultative anaerobic bacteria were then encapsulated in aqueous droplets using a 6-junction droplet chip (DOLOMITE® microfluidics). The droplet chip produces aqueous droplets of a chosen growth media (such as Brain Heart Infusion (BHI) medium used in this example), which is surrounded by an oil-phase (fluoro-carbon oil, BIORAD®). The droplets were incubated in growth media at 37° C., under aerobic conditions for ˜12 hours. After incubation, the droplets demonstrated five distinct bacterial morphologies within droplets labelled A-E. (FIG. 9). These results also demonstrate the ability to isolate bacteria from a mixed community into individual droplets. (FIG. 9).

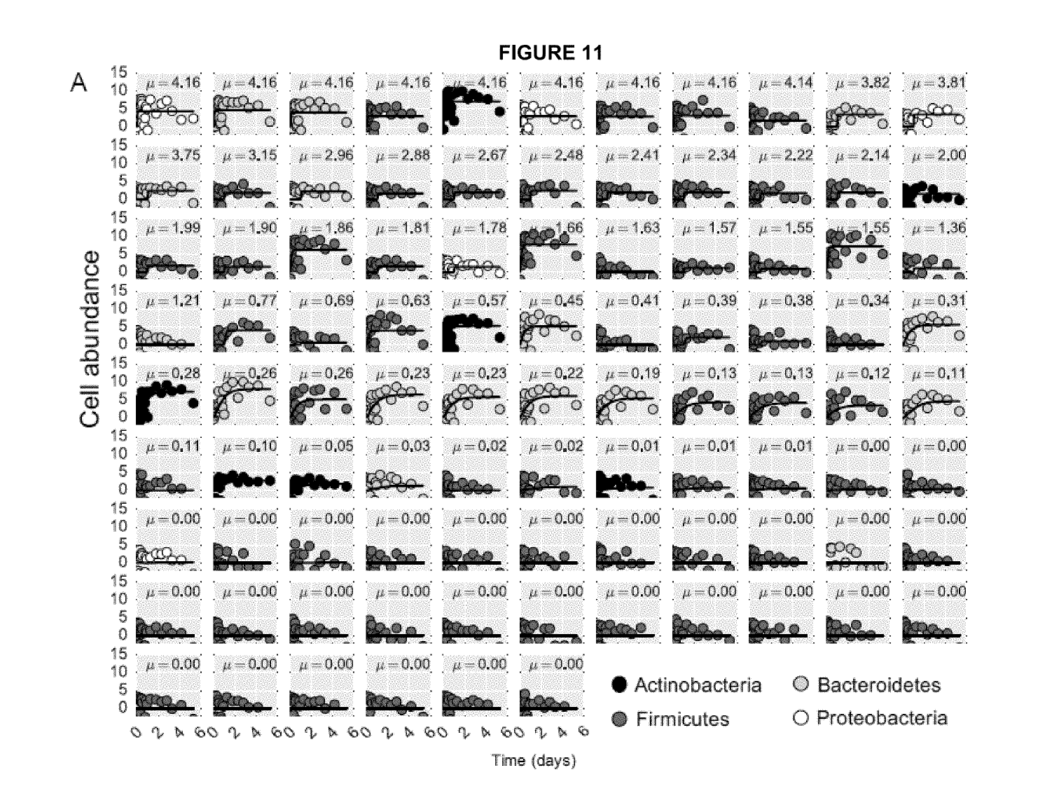

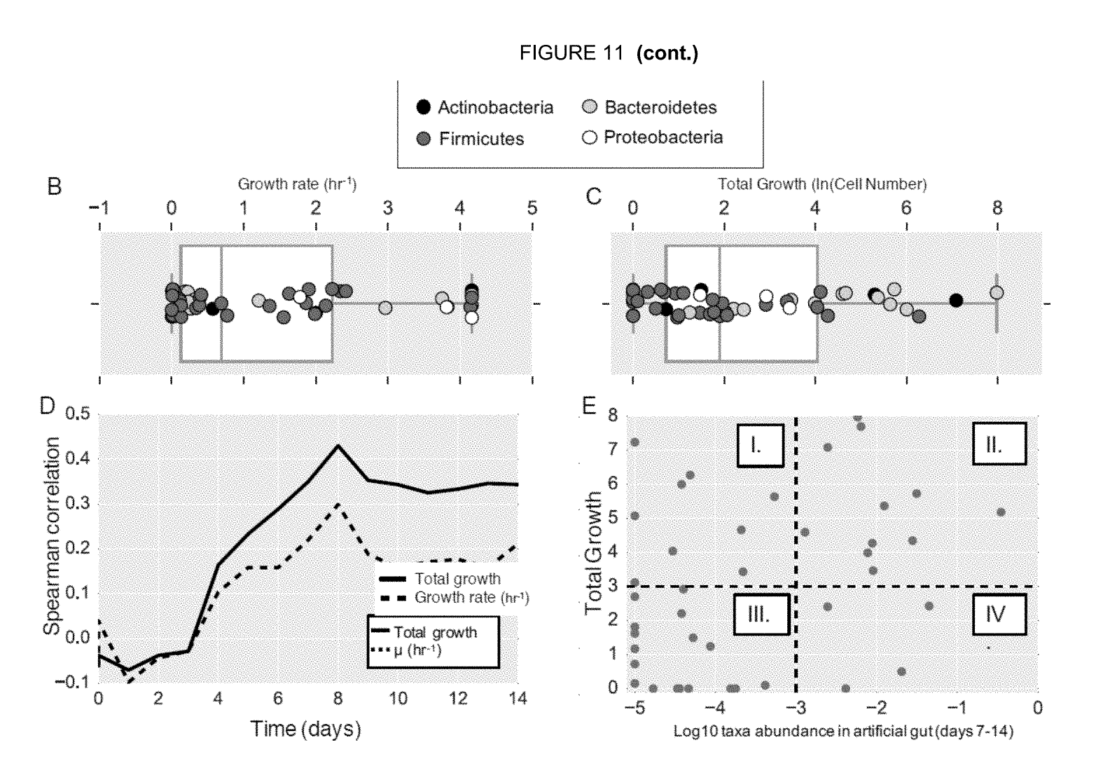

(B) Next generation DNA sequencing combined with quantitative PCR (qPCR) allows for characterizing absolute numbers of bacteria isolated in droplets. Once encapsulated, the bacterial droplet cultures are incubated under conditions appropriate for growth. At a set time point, commonly T0 (right after droplet making) and T24 (after 24 hours of incubation), DNA is extracted from the droplets using a standard extraction kit. Relative abundances of different taxa are determined by sequencing using the 16S V4 rRNA primers (GTGCC AGCMG CCGCG GTAA (SEQ ID NO: 1) and GGACT ACHVG GGTWT CTAAT (SEQ ID NO: 2)). The amount of DNA present is measured by quantitative qPCR, also using the 16S V4 rRNA primers (SEQ ID NO: 1 and SEQ ID NO: 2). To compute absolute growth of an individual bacterial strain within a complex microbial community, the quantitative qPCR value is multiplied by the relative abundances from sequencing. In this way, the absolute growth of an individual bacterial strain can be determined directly from an environmental community (such as a fecal sample) in a specific condition. (See FIG. 11A-E).

### Example 3: Bacteria Grown in Droplets Recapitulate Total Growth of Gut-Isolated Bacteria Compared with a Plate Reader

To assess the ability to accurately measure the growth of bacteria isolated from a mixed community, bacteria isolated from the gut were grown in both a plate reader and droplets. To generate droplet cultures, nine individual isolates of bacteria from the human gut were grown in culture overnight in Gifu Anaerobic media with the addition of vitamin K and Hemin (modified Gifu Anaerobic media; mGAM). Cultures were grown anaerobically at 37° C. The nine cultures were then mixed together and diluted to isolate only one bacterium per droplet. The mixed culture had an OD600 of 0.66, and 8.7 μl of the mixed culture was added to 4 mL of mGAM medium. Droplets were generated using a 6-junction droplet chip (DOLOMITE® microfluidics), flowing mGAM medium through the oil (fluoro-carbon oil, BIORAD®). Droplets were sampled at the time of generating the droplets (T0), and the remaining droplets were incubated anaerobically at 37° C. for 24 hours. Simultaneously with generation of the droplet cultures, bacterial isolates were diluted 1:20 in mGAM medium (10 μl of overnight culture into 190 μl of mGAM medium in a well) and put into 96-well plates to assess their growth individually. To assess the growth of the isolates individually, OD600 was taken at T0 and again at T24, after incubating the 96-well plates anaerobically at 37° C., as shown in Table 1 below.

TABLE 1
Growth of isolates measured by plate reader (control).

| | | Control |
|---|---|---|
| | Control T0 | T24 |
| Isolate | (OD600) | (OD600) |
| Bacteroides fragilis (Bfs1) | 0.038 | 1.094 |
| Bacteroides thetaiotamicron (Bta) | 0.198 | 1.973 |
| Shigella flexineri (Sfi) | 0.317 | 1.163 |
| Bacteroides vulgatus (Bvg) | 0.355 | 0.613 |
| Streptococcus salivarius (Sss) | 0.203 | 1.7215 |
| Bacteroides caccae (Bce) | 0.3355 | 1.066 |
| Bifidobacterium longum (Blm) | 0.136 | 0.3115 |
| Bacteroides uniformus (Bus) | 0.0885 | 1.6505 |
| Clostridium inocuum (Cim) | 0.273 | 0.114 |

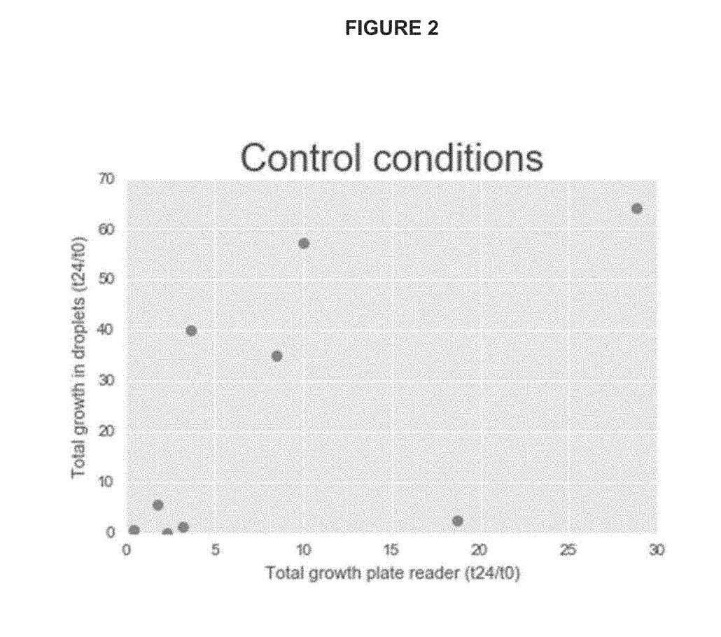

Absolute growth of the bacteria from droplets was measured by sequencing. Using a standard extraction kit, DNA was extracted from the samples taken at T0 and T24. qPCR was used to quantify the total amount of DNA by measuring 16S V4 rRNA at each time point, using the 16S V4 rRNA primers (GTGCC AGCMG CCGCG GTAA (SEQ ID NO: 1) and GGACT ACHVG GGTVVT CTAAT (SEQ ID NO: 2)). Growth was determined by dividing the absolute amount of each isolate at T24 by the absolute amount at T0. The absolute growth in droplets was then compared to the equivalent measure of growth of the plate reader, which was determined by taking growth measurements (OD600) at T0 and T24, and dividing the OD600 at T24 by the OD600 at T0. This comparison demonstrated a strong correlation between the droplet measurements and the plate reader measurements (Spearman rho: 0.70, pvalue=0.04). (FIG. 2). These results indicate that droplets are a valid way to assay growth of individual isolates.

### Example 4: Antibiotic Assays in Droplets Using Endpoint Measurements

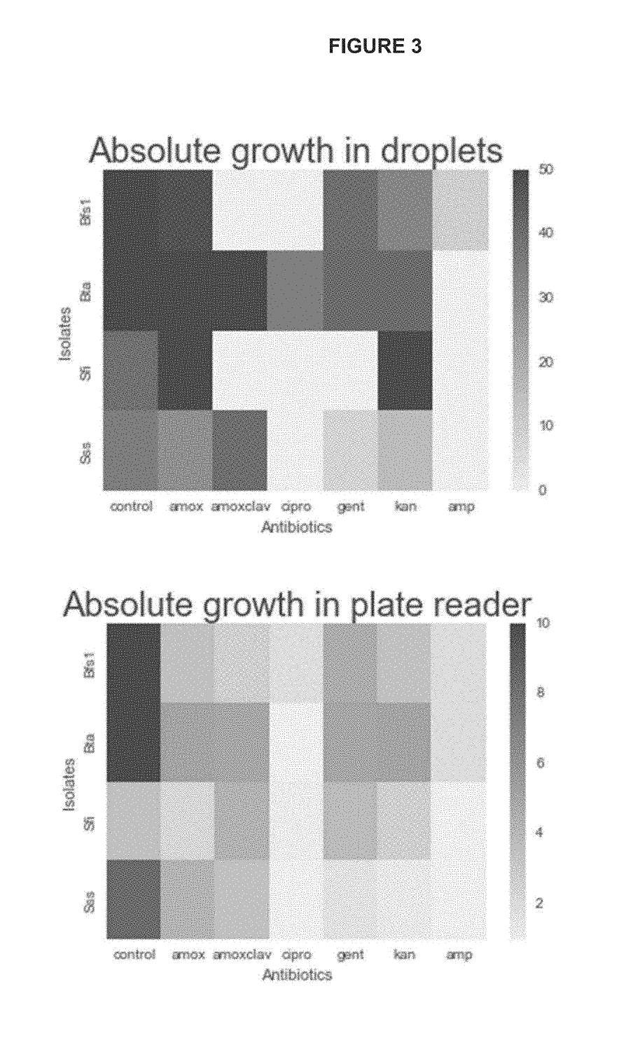

Bacteria from the human gut were isolated and cultured under conditions appropriate for growth. The cultures were then mixed together to form an artificial mixed community and diluted to isolate one bacterium per droplet in media with various antibiotics and without (control). Droplets were generated by the method in Example 2. Droplets were sampled at the time of generating the droplets (T0), and the remaining droplets were incubated anaerobically at 37° C. for 24 hours. DNA was extracted from the samples taken at T0 and T24, and total DNA was quantified by qPCR as provided in Example 2. Simultaneously, prior to mixing the isolated cultures, bacterial isolates were added to a plate reader in the presence of a respective antibiotic to compare with growth of the same isolated bacterial strain in droplets in the presence of a respective antibiotic. (FIG. 3). Absolute growth was computed by taking the absolute number of cells at T24 and dividing that by the absolute number of cells at T0. (FIG. 3).

Only isolated bacteria which demonstrated growth in droplets under the control condition (without antibiotics) were utilized in this assay, because of the inability to determine whether failure of an isolated bacteria to grow in droplets under the control conditions is attributed to sensitivity to an antibiotic or inability to grow in the droplet environment. There may be multiple reasons why a bacterial isolate does not grow in droplets such as, for example, suitability of media or a lack of a solid substrate. After analyzing the bacteria that were able to grow in droplets, the threshold for sensitivity to a respective antibiotic was determined to be a bacterial isolate cultured with a respective antibiotic that exhibited less than 50% of its growth in the control. Using this criteria and the growth in the plate reader as the standard value for antibiotic susceptibility, FIG. 3 shows that growth in droplets correctly predicted the antibiotic sensitivity of 21 out of 28 combinations (accuracy of 75%). (FIG. 3).

### Example 5: Measuring Growth Rates from a Full Fecal Community in Droplets

Bacteria present in a stool sample were isolated in droplets, according to the methods detailed in Example 1. A majority of the bacteria from the human gut microbiota that are present in stool were captured. This data is presented in the genus column for control_b and control_d entries, showing that of the bacteria present within the original fecal community (i.e., control_d), over 84% of the genera were grown in droplets. This is in contrast with the control_b that shows in a traditional batch culture (all bacteria grown together, no isolation within droplets), only 42% of the genera were able to grow. The subsequent rows provide growth under other conditions that were conducted on the full community, all grown in droplets. 02=grown in the presence of oxygen, ph=grown at a pH of 5.5 (normal media is pH 7), salt=grown with 2.5% salt, temp=grown at 39° C. (compared with 37° C.). These conditions were chosen as biologically relevant conditions that exist in gradients along the human gastrointestinal tract.

TABLE 2

| | Kingdom | Phylum | Class | Order | Family | Genus | otus | |
|---|---|---|---|---|---|---|---|---|
| control_b | 0.5 | 0.666667 | 0.7 | 0.636364 | 0.652174 | 0.424242 | 0.238245 | Batch |
| | | | | | | | | culture |
| control_d | 0.5 | 0.833333 | 0.9 | 0.818182 | 0.913043 | 0.848485 | 0.620690 | Droplet |
| o2 | 1 | 1 | 1 | 0.909091 | 0.956522 | 0.909091 | 0.642633 | culture |
| ph | 1 | 1 | 1 | 0.909091 | 0.956522 | 0.878788 | 0.673981 | |
| salt | 1 | 1 | 1 | 0.909091 | 0.913043 | 0.818182 | 0.539185 | |
| temp | 0.5 | 0.833333 | 0.9 | 0.818182 | 0.826087 | 0.757576 | 0.570533 | |

Growth Rate Data from Full Fecal Community

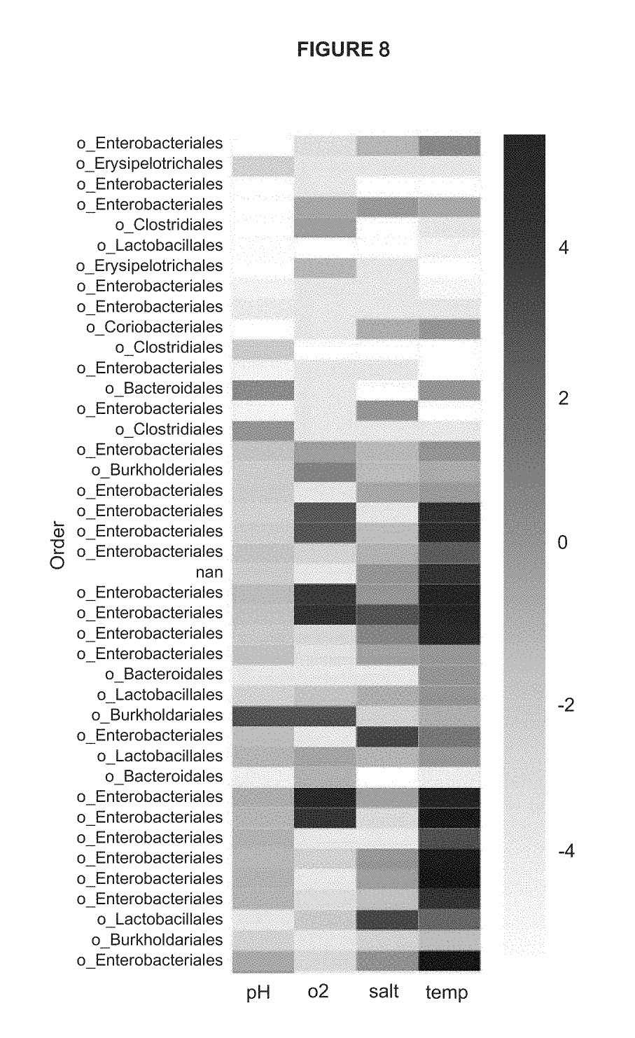

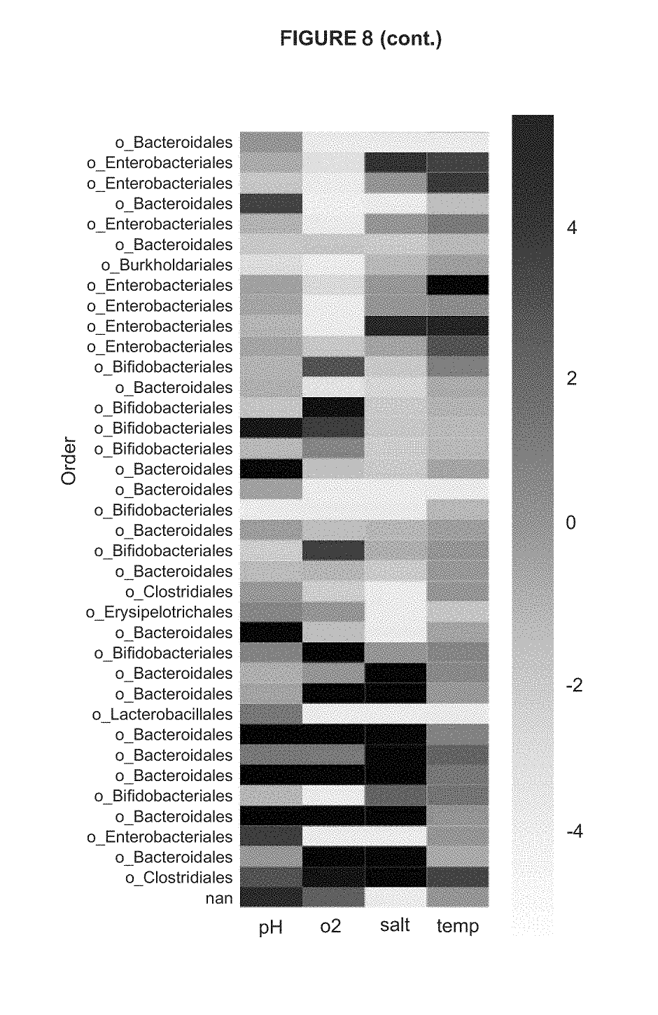

FIG. 8 provides growth rate data from a full fecal community, relative to the control growth rate under each of the following conditions: o2=grown in the presence of oxygen, ph=grown at a pH of 5.5 (normal media is pH 7), salt=grown with 2.5% salt, temp=grown at 39° C. (compared with 37° C.). The darker black indicates a faster growth rate under those conditions, while the lighter white indicates that the growth rate was slower at that condition than in the control. Light grey boxes indicate missing values, or that there was no growth for that individual bacterial order under the corresponding condition. Each row indicates individual bacteria that are labelled at the order level. The data from FIG. 8 is summarized in Table 2, which shows that most of the taxa lost are lost due to growth in the presence of oxygen (# taxa lost). This is not unexpected, because the large intestine (where most of the gut bacteria are found) is known to be anaerobic, and many of these bacteria are known to be obligatory anaerobes.

TABLE 3

| | | # taxa lower | # taxa higher |
|---|---|---|---|
| condition | # taxa lost | growth rate | growth rate |
| pH 5.5 | 3 | 58 | 16 |
| o2 | 25 | 30 | 22 |
| 2.5% salt | 18 | 39 | 17 |
| 39° C. (temp) | 7 | 30 | 36 |

### Example 6: Growth Response to Xenobiotics

To determine gut bacterial strain response to xenobiotics, gut bacteria were screened for beta-glucuronidases, which are present in bacteria that can inactivate certain man-made therapeutics. Results of the screen show bacterial growth on the monosaccharides glucose and glucuronic acid, as well as a conjugated form of glucuronic acid: 4-Nitrophenyl-Beta-D-glucuronic acid (PNPG). (FIG. 6). Bacterial growth on PNPG requires beta-glucuronidase (4-Nitrophenyl-Beta-D-glucuronic acid) to liberate glucuronic acid bound to the phenol group of PNPG. Thus, the assay identified bacterial taxa that contain putative beta-glucuronidases. Glucose and water are positive and negative controls, respectively. (FIG. 6).

### Example 7: Growth Assays in Droplets Using a Defined Medium and Only One Time Point

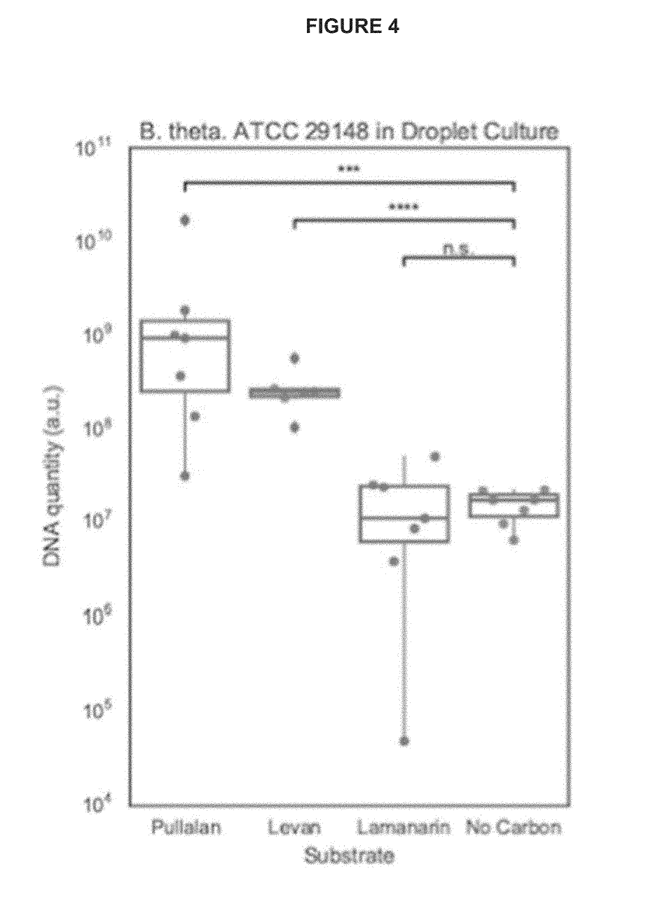

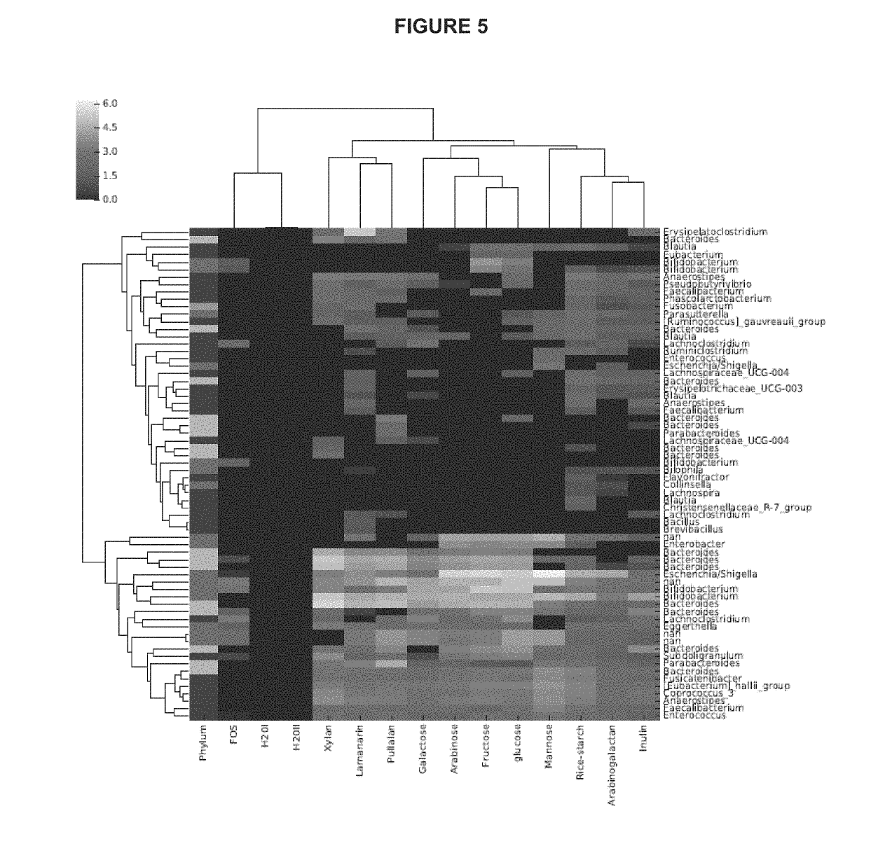

Bacterial growth on a defined medium is an important tool for phenotyping. By using a defined media, a function of individual bacteria (in this case growth on a substrate of interest) can be determined. Bacteria were isolated from a fecal community of a healthy donor and cultured in defined medium. To identify carbon sources on which an individual isolated bacterium can grow, carbon sources of varying complexity were added individually to the defined media. Growth was measured at the endpoint, and the DNA was extracted, sequenced and quantified by qPCR as provided in Example 2. In this assay, the endpoint growth of an individual bacteria cultured in a defined medium with the addition of one carbon source was compared to the end point growth measurement of the same bacteria in the defined medium with only the addition of water. (FIG. 4). This comparison enables assessing the ability of an individual microbe to use a single carbon source for growth. As an example in FIG. 4, an isolate of the bacterial strain B. thetaiotamicron (ATCC® strain 29148) was cultured in droplets with defined medium containing the carbon source Pullalan, Levan, Lamanarin or no carbon source (control), and growth was compared. (FIG. 4). The results show that, compared to the control defined medium without the addition of a carbon source, the B. theta isolate exhibited greater growth under carbon sources Pullalan and Levan, while growth of the isolate under carbon source Lamanarin was the same or slightly worse than growth under the control. (FIG. 4). FIG. 5 shows high throughput measurement of growth of gut bacteria isolated from a fecal community of a healthy donor, each of which were cultured in droplets containing defined media with the individual addition of various dietary monosaccharides and polysaccharides. (FIG. 5).

### Example 8: Differential Growth Responses to Amino Acid Supplementation

Using the defined medium, growth of bacterial isolates was assessed in the presence of limited nutrient conditions, such as amino acid supplementation. For example, growth of bacteria with and without amino acid supplementation can be compared. Bacteria that increase in growth when the amino acid is present are consumers of the amino acid. The role that microbiota play in amino acid metabolism is thought to play a role in human metabolic syndrome. FIG. 7 compares growth of putative amino acid consumers from a healthy individual to two individuals with metabolic syndrome. Here, growth of various individual bacterial species is compared when grown on defined medium in the presence of isoleucine. (FIG. 7). Growth was determined by comparing an endpoint measurement with growth of the same bacteria on media without the isoleucine supplement. (FIG. 7). Growth of different bacteria between different individuals demonstrated different responses to the same amino acid. (FIG. 7). Accordingly, this tool can be used to compare the function of microbial communities between individuals, either healthy or those exhibiting metabolic syndrome.

### Example 9: Growth Dynamics of Human Gut Microbiota

To estimate SV growth curves using microfluidic droplets (“MicDrop”), a total of 70 separate microfluidic droplet aliquots were collected for destructive longitudinal sampling. Droplets were generated according to the MicDrop protocol described above. A modified Gifu Anaerobic Medium (mGAM) was used in the droplets (Gifu Anaerobic Medium, HIMEDIA®, with the addition of 5 mg/L Vitamin K and 10 mg/L Hemin). Each aliquot of 200 μl of droplets was incubated at 37° C. in an anaerobic chamber. Aliquots were destructively sampled in triplicate, hourly, for hours 0-24 after droplet making and in duplicate once a day for hours 24-127 after droplet making.

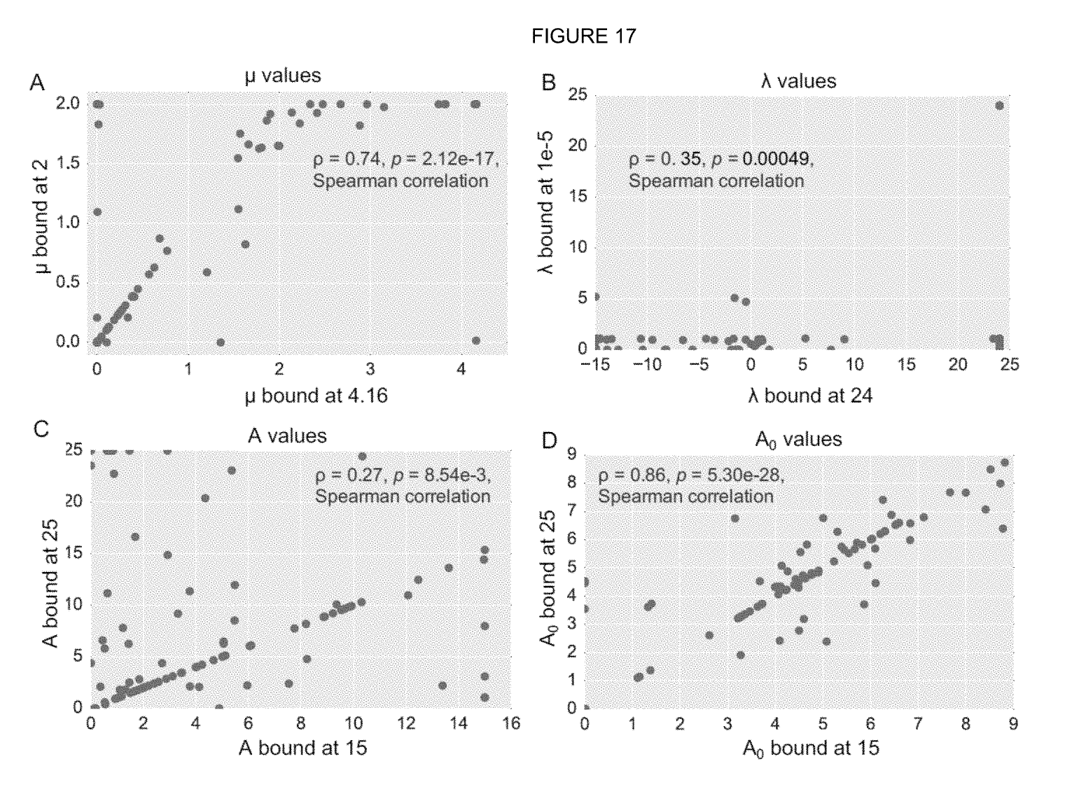

Growth curves were fit using a combination of 16S rRNA qPCR and DNA sequencing data. To minimize the potential for poorly fit growth curves, SVs were required to have been detected by DNA sequencing in >5 samples to be included in curve fitting. To avoid numerical instabilities associated with taking the log or dividing by zero, a pseudocount of one was added to the sequence variant count table prior to normalization to relative abundances. Relative abundances of each SV were then determined by dividing the number of counts associated with each taxon in each sample by the total read counts in the sample. Concentrations of each taxa were then estimated by multiplying the relative abundances of SVs by the 16S rRNA concentrations determined by qPCR. Technical replicates constituted distinct data points in these calculations. The SciPy Python package (v0.19.1) was used to fit a modified Gompertz equation to which we added an additional term to account for differences in starting abundance to the resulting dataset: y=Aexp{−exp[(μ·e)/A (λ−t)+1]}+A_0, where μ is growth rate, A is carrying capacity, λ is lag time, or the time it takes for a bacteria to reach logarithmic growth, and A₀ is starting abundance. Curves were fit using the module scipy.optimize.curve_fit and the Trust Region Reflective algorithm (“trf”). Parameter bounds were also used to minimize the optimization search space. Lower perimeter bounds of A=0, λ=−15, μ=0, A₀=0; and, upper perimeter bounds of A=15, λ=24, μ=4.16, A₀=15) were set. Curve fits were initialized by setting variables to the lower bound. The inventors chose to bind μ at 4.16, representing a doubling time of 10 minutes, as this is considered one of the fastest growth rates observed in a bacterium. The upper bound on A and A₀ was based on the absolute values from qPCR and the inferred carrying capacity of the droplets. The upper bound on λ was chosen to allow for acclimation of the droplet environment. Sensitivity analysis indicated that all parameters were most sensitive to the choice of bounds, particularly the choice to bind μ (FIG. 17). Growth rates (μ) were converted to doubling times using the equation: ln(2)/μ.

### Example 10: MicDrop Prebiotic Assay and Validation of Prebiotic Utilization Assays

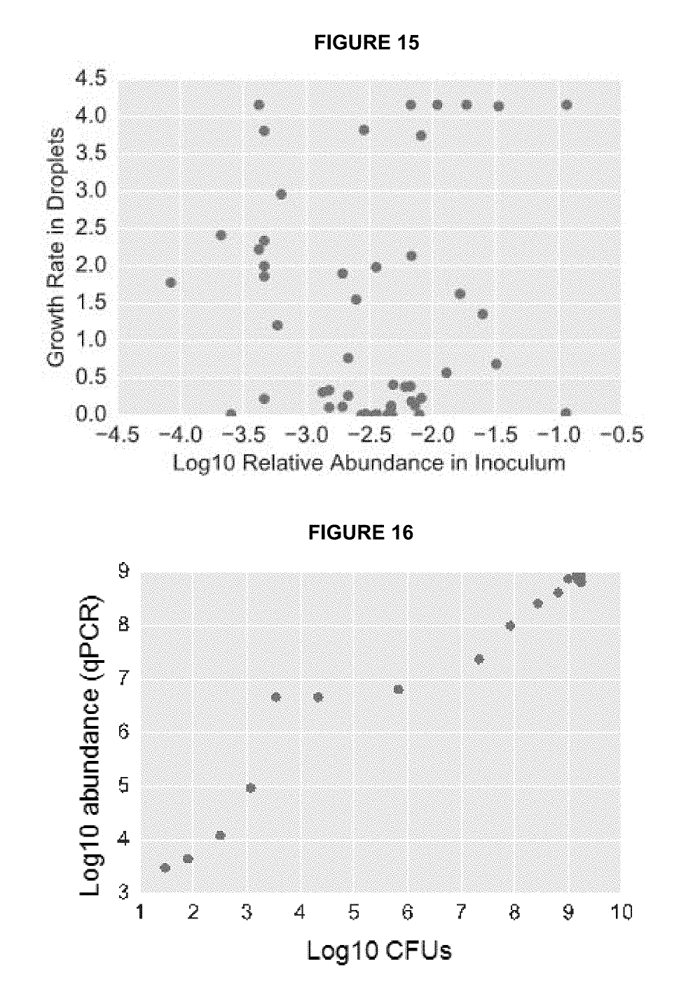

(A) Microbial communities isolated from human stool samples were examined for carbon consumption in a custom microfluidic screen. Cells were restarted from frozen stock in mGAM (as above) and then minimal medium containing glucose and galactose (SIGMA®) as the sole carbon sources. Following determination of the loading concentration, the cell culture stocks were washed twice by centrifugation and resuspended in 2× minimal medium. Cells were filtered using a 50 μm filter (CELLTRICS®, SYSMEX®) to remove multi-cell clumps. The filtered cells suspension was then added to each sample formulation at the target concentration. Each sample was a 50:50 mixture of 1% carbon solution and 2× minimal medium. To prevent chip fouling, the oil inlet was equipped with 10 μm inline filters (P-276, IDEX®). Droplet generation in the anaerobic chamber was monitored using a bright field microscope (44347, CELESTRON®). Droplet cultures were stored 5 mL polypropylene tubes (352063, FALCON®) with the caps closed in an anaerobic incubator at 37° C. Following the second day of incubation, cultures were moved to a −20° C. freezer for storage prior to DNA extraction. Pre-processing of the droplet data designated samples with qPCR values below the mean no-carbon control as zero growth. Then, relative abundance data was converted to absolute abundance by multiplying each sample by the corresponding qPCR value. The median was then calculated across three replicate samples and matched no-carbon controls were subtracted from each sample. Finally, a growth threshold of 88% of maximum growth was applied, as determined from validation analysis (FIG. 15, Validation of prebiotic utilization assays)

(B) The MicDrop prebiotic assay was validated by generating reference data on carbohydrate preferences using a synthetic community of seven wild type gut isolates from an in-house collection (Table 4) in 96-well plates following the same procedure described for the MicDrop prebiotic assay. Briefly, 96-well plates were prepared with minimal medium (Table 5) and a carbohydrate as a sole carbon source in a mixture that had equilibrated in anaerobic conditions overnight. Prior to loading into 96-well plates, bacterial cells were pre-cultured using an overnight culture in minimal medium containing glucose and galactose, then washed twice by centrifugation (2 min at 14,000 g) to remove free monosaccharides, and finally resuspended in minimal medium without a carbon source. A 10 μL aliquot of cell suspension was added to 200 μL of medium in 96-well plates and incubated in a humidified container for two days at 37° C. All culture experiments were performed in an anaerobic chamber. Following the culture period, the optical density at 600 nm of each well was examined using a plate reader (CLARIOstar®, BMG LABTECH®). Following published protocols, growth in plates was normalized to the maximum growth on the preferred carbon source for each microbe. Then, to yield binary “growth” or “no-growth” data, a threshold of 20% of maximum growth was applied to the plate data, above which was considered growth on the carbon source of interest. The same synthetic community and carbon sources were then examined using the MicDrop prebiotic assay described above. To determine the optimal growth threshold to apply to the droplet data, Youden's J index across all possible threshold values was calculated, with the maximum value of Youden's J corresponding to a droplet threshold of 88% of maximum growth.

TABLE 4
List of strains used in validation experiments.

| Species | Usage | Source |
|---|---|---|
| Bacteroides thetaiotaomicron | Prebiotic monoculture | ATCC |
| ATCC 29148 | validation | |
| Bacteroides ovatus | Prebiotic synthetic | Fecal isolate |
| | community | |
| Ruminococcus gnavus | Prebiotic synthetic | Fecal isolate |
| | community | |
| Escherichia coli | Prebiotic synthetic | Fecal isolate |
| | community | |
| Klebsiella granulomatis | Prebiotic synthetic | Fecal isolate |
| | community | |
| Bacteroides vulgatus | Prebiotic synthetic | Fecal isolate |
| | community | |
| Enterococcus faecalis | Prebiotic synthetic | Fecal isolate |
| | community | |
| Bacteroides 53121 | Prebiotic synthetic | Fecal isolate |
| | community | |
| NDL-177, Eschericia coli | Fluorescent droplet | Gift from N. Lord |
| | experiments | |
| Streptococcus agalacticaeae | Validation synthetic | Fecal Isolate |
| | community | |
| Bacteroides fragilis | Validation synthetic | Fecal Isolate |
| | community | |
| Bifidobacterium longum | Validation synthetic | Fecal Isolate |
| | community | |

TABLE 5
Minimal Medium Formulation
Recipe to make 2X Minimal Medium to be added to 2X carbon stock.
Pre-made vitamin, trace elements and amino acid solutions
were incorporated to enhance reproducibility.

| Component | 2X Concentration (M) | Source |
|---|---|---|
| Monopotassium phosphate | 2.0e−1 | Sigma |
| Sodium chloride | 3.0e−2 | Sigma |
| Ammonium sulfate | 1.7e−2 | Sigma |
| L-cysteine | 8.0e−3 | Sigma |
| Hematin | 3.8e−6 | Sigma |
| L-histidine | 4.0e−4 | Sigma |
| Magnesium chloride | 2.0e−4 | Sigma |
| Iron (III) sulfate | 2.8e−6 | Sigma |
| Calcium chloride | 1.0e−4 | Sigma |
| Menadione | 1.2e−5 | Sigma |
| Cobalamin | 8.0e−9 | Sigma |
| Vitamins and Minerals | 2X working concentration | ATCC |
| Trace Elements | 2X working concentration | ATCC |
| Amino Acid Supplement | 2X working concentration | Sigma |
| | | AA-5550 |
| Purine and Pyramidine solution | 2X working concentration | Sigma |
| Sodium hydroxide to pH 7.0 | — | |

### MicDrop Prebiotic Assays Using Human Stool Samples

Stool samples were collected from nine healthy donors (7 men, 2 women) between the ages of 35-53 under the IRB protocol described above. To facilitate carrying out prebiotic assays simultaneously across a range of donors, we used frozen gut microbiota in these experiments. Fecal slurries were made at 10% w/v using mGIFU medium and a stomacher (SEWARD®) that homogenized fecal samples for one minute. Then, slurries were mixed 50:50 with 50% glycerol and stored at −80° C. for later use. Finally, the samples were assayed following the MicDrop prebiotic assay procedure described above.

### Example 11: Identifying Primary Degraders from Human Guts Across Multiple Prebiotics

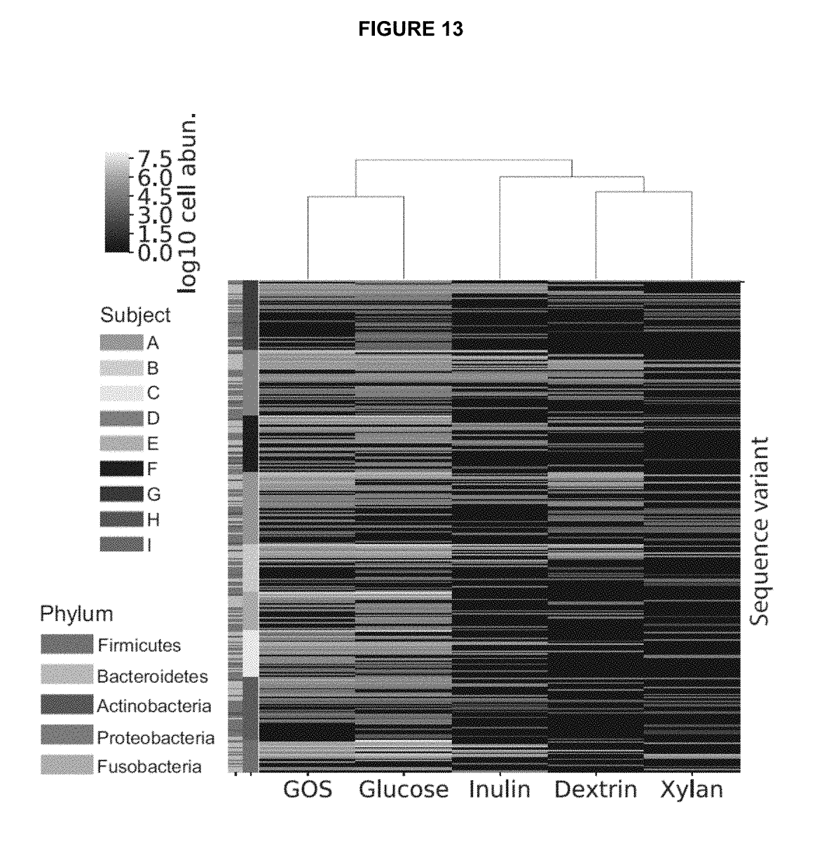

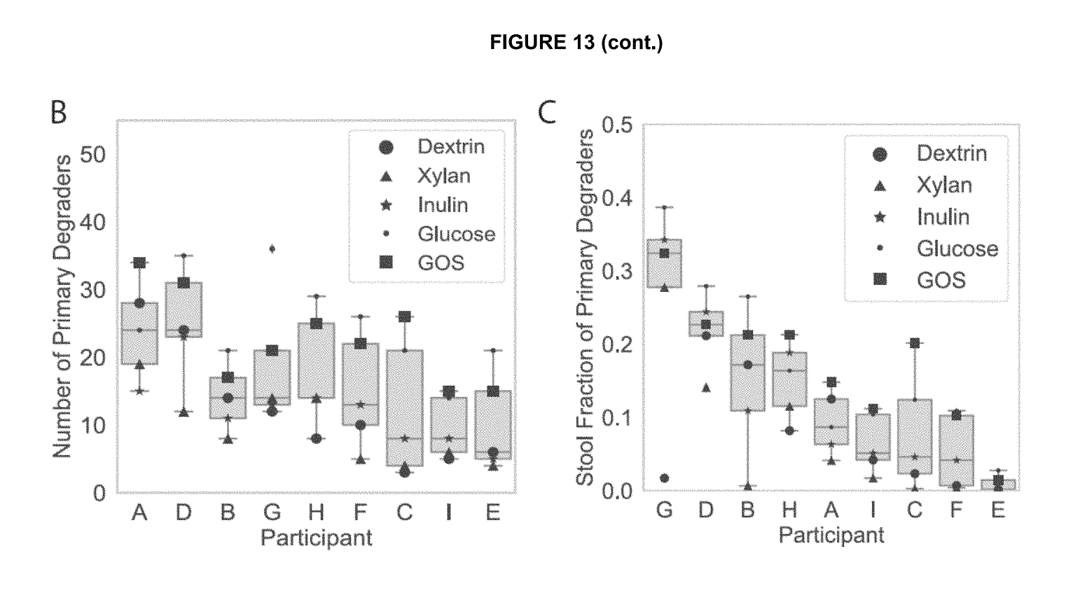

The MicDrop prebiotic assay was applied to microbiota from nine healthy human stool donors. Growth was assayed using three commercially available prebiotics (inulin, galacto-oligosaccharides (GOS), and dextrin) and a lab-grade prebiotic (xylan). Out of the 588 SVs detected across donor stool samples, MicDrop identified a total of 293 primary degraders that grew on at least one of the screened prebiotics (FIG. 13A). These primary degraders were similar to those found in a culture-based database of bacterial carbohydrate utilization, which were used as a gold-standard for whether a primary degrader could consume a given prebiotic. Of the 46% of primary degraders with annotations in this database, a true positive rate of 86% and an overall assay accuracy of 87% was computed (Table 6). Extrapolating this true positive rate to the 75 primary degraders that did not have annotations in the database, the MicDrop assay yielded prebiotic utilization profiles for roughly 64 primary degrading taxa that have not been previously annotated.

However, differences in the presence or absence of primary degraders do not appear to drive inter-individual variation in human prebiotic response. Instead, it was observed that multiple SVs capable of growing on the tested prebiotics were present in all subjects (median: 12.5±6.8); (FIG. 13C, Table 6). Additionally, primary degraders were more likely to be shared between individuals than SVs not identified as primary degraders in stool samples (p=1.22e-3, Chi-square). Concordant with human studies demonstrating that even subjects with low prebiotic fermentation in vivo had at least some prebiotic fermentative capacity in vitro, these findings suggest that primary degraders are likely widespread across individuals.

Inter-individual variation in primary degrader composition and abundance was identified. Differences were observed in the richness of primary degraders across subjects (p<0.001, Two-way ANOVA; FIG. 13C). Subject identity explained more variation (R2=0.30, PERMANOVA; Table 7) than prebiotic type (R2=0.16, PERMANOVA; Table 7) in overall primary degrader growth. Last, differences in the relative abundance of primary degraders were observed in inoculating fecal communities (p<0.0001, Two-way ANOVA; FIG. 13D). Thus, while primary degraders are likely present in most individuals, differences in polysaccharide metabolism may be due to inter-individual variation in primary degrader activity and abundance.

TABLE 6
Binary Classification Metrics of MicDrop prebiotic assay performance
compared to reference bacterial carbohydrate utilization profiles

| Sample | Accuracy | TPR | TNR | Precision | FPR | FNR | FDR | MCC |
|---|---|---|---|---|---|---|---|---|
| Primary degraders | 0.87 | 0.86 | 0.87 | 0.52 | 0.12 | 0.14 | 0.48 | 0.60 |
| from 9 healthy | | | | | | | | |
| subjects | | | | | | | | |
| Permuted data | 0.77 | 0.37 | 0.86 | 0.37 | 0.14 | 0.63 | 0.63 | 0.24 |
| (median of 1000 | | | | | | | | |
| permutations) | | | | | | | | |
| Fraction of 1000 | 1.00e−3 | 0.00 | 0.41 | 9.70e−2 | 0.10 | 0.00 | 0.10 | 1.00e−3 |
| permutations equal | | | | | | | | |
| to or better than | | | | | | | | |
| observed data | | | | | | | | |

TABLE 7
PERMANOVA of primary degraders by subject and prebiotic.

| | DF | Model F | R² | p |
|---|---|---|---|---|
| Subject | 8.00 | 6.81 | 0.30 | 0.001 |
| Prebiotic | 3.00 | 9.65 | 0.16 | 0.001 |

Microfluidic investigation of prebiotic response reveals the breadth of taxa that respond to carbohydrates and supports hypotheses that inter-individual variation to carbohydrate interventions is due to differential abundances of polysaccharide degrading bacteria between people. In some aspects, this disclosure provides that droplet microfluidics could be used in the future to stratify human populations into groups most likely to benefit from prebiotic treatments, by providing a culture-based diagnostic approach capable of scaling to the diversity of microbes inhabiting the human gut.

### Comparison of Primary Degraders to the Virtual Metabolic Human Database of 719 Microbes.

The identity of primary degraders was compared to carbon consumption profiles from the Virtual Metabolic Human (VMH) database composed of curated culture-based data for 719 microbes. Primary degraders were first mapped to this database by taking SV 16S rRNA sequences from these studies and BLASTing them against the NCBI database. Each 100% match was then linked to a type strain in the VMH database using NCBI taxonomy ids. A NCBI genome assembly file (ftp://ftp.ncbi.nlm.nih.gov/genomes/ASSEM BLY_REPORTS/assembly_summary_genbank.txt) was used to translate between species level and strain level taxonomy ids in the VMH database. Then, only SVs where all blast matches to the VMH database that were uniformly true or false prebiotic consumers in the VMH database were tabulated. Only xylan and inulin prebiotics were examined since the other carbon sources were not part of the VMH database. Permutation analysis were carried out by randomly shuffling rows and columns of the droplet primary degrader table and compared to the VMH database. Then the median of 1000 permutations was tabulated.

### Example 12: Prebiotic Consumption by Human Gut Microbes

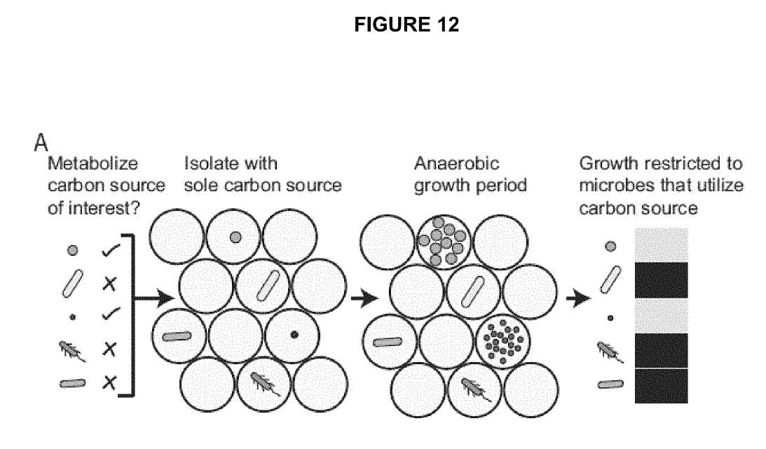

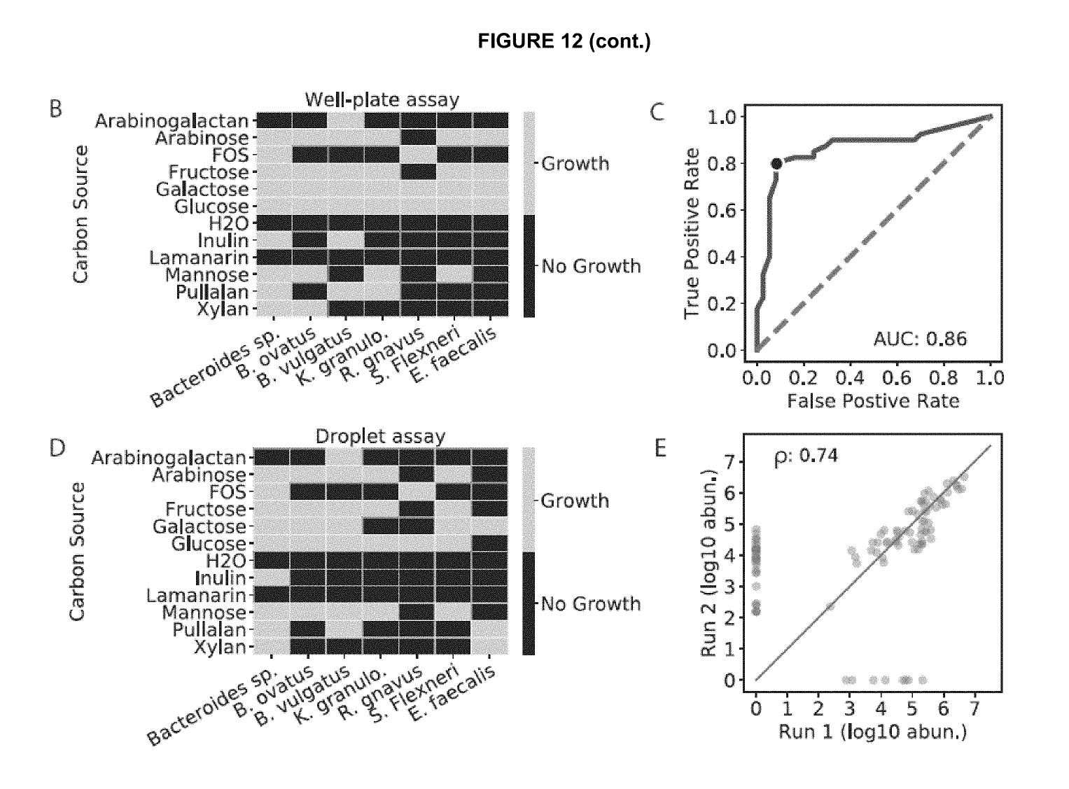

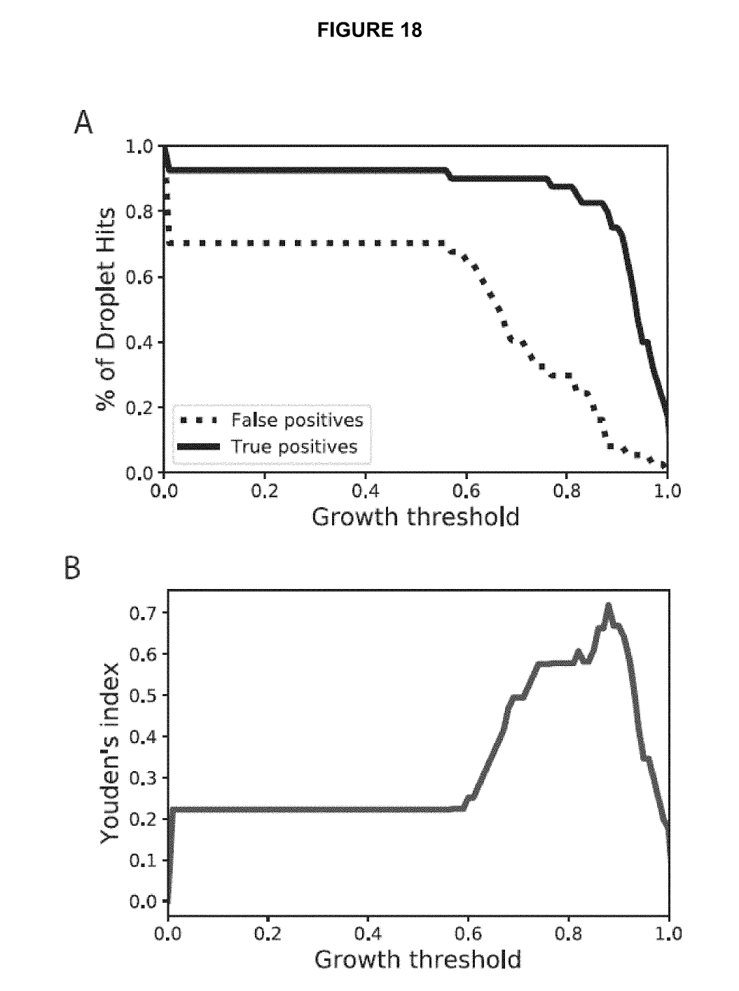

To test whether the MicDrop method could be used to assay bacterial prebiotic metabolism (FIG. 12A), a previously characterized type strain Bacteroides thetaiotaomicron ATCC 29148 was loaded into microfluidic droplets and standard 96-well plates. Consistent with both prior studies and well-plate experiments disclosed herein, B. thetaiotaomicron ATCC 29148 grew in droplets on pullulan and levan, but not on lamanarin or a no carbohydrate control (FIG. 4). Next, performance of the MicDrop prebiotic assay was tested using synthetic microbial communities assembled from seven human gut isolates (FIG. 12C). Using 96-well plate experiments as a reference (FIG. 12B, FIG. 18), the accuracy of the MicDrop prebiotic assay was determined to be 87% (Table 8). Finally, to assess the reproducibility of the MicDrop prebiotic assay, the same frozen fecal sample was used to compare the assay between two separate experimental sessions. A higher correlation was observed between replicates from the same session (p=0.73-0.77, p<0.0001, Spearman correlation) than between replicates across sessions (p=0.57, p=1.07e-17, Spearman correlation; FIG. 12E). This difference in correlation was likely due to stochasticity in microbiota composition after frozen stool was revived for droplet inoculation, as controlling for microbiota differences between droplet inocula elevated the between session correlation (p=0.74, p=1.50e-19, Spearman correlation).

TABLE 8
Artificial community binary classification performance metrics.

| Sample | Accuracy | Sensitivity | Specificity | Precision | FPR | FNR | FDR | MCC |
|---|---|---|---|---|---|---|---|---|
| Artificial | 0.869 | 0.800 | 0.931 | 0.914 | 0.068 | 0.200 | 0.085 | 0.741 |
| Community | | | | | | | | |

TABLE 9
Disclosed Nucleic Acid Sequences

| | |
|---|---|
| SEQ ID NO: 1 | Forward primer sequence for 16S V4 |
| | rRNA |

GTGCCAGCMGCCGCGGTAA

| | |
|---|---|
| SEQ ID NO: 2 | Reverse primer sequence for 16S V4 |
| | rRNA |

GGACTACHVGGGTWTCTAAT

What is claimed is:

1. A method for measuring absolute growth of a bacterial strain from a mixed microbial community, comprising:
(a) isolating a single bacterium from a mixed microbial community by encapsulating the bacterium in an aqueous droplet surrounded by an oil-phase to obtain an encapsulated bacterium;
(b) incubating the encapsulated bacterium under conditions appropriate for growth to obtain a single encapsulated bacterial strain;
(c) extracting DNA from the encapsulated bacterial strain at one or more time points during incubation;
(d) measuring:
(i) the quantity of total DNA extracted at each time point using quantitative Polymerase Chain Reaction (“qPCR”) via primers that target a variable region within a conserved gene sequence; and
(ii) the relative abundance at each time point of the encapsulated bacterial strain in the mixed microbial community through sequencing of the same variable region within a conserved gene sequence as in (i) using the same primers as in (i); and
(e) determining absolute growth based on the measurements obtained in (d) at each time point.

2. A method for characterizing the microflora in a patient's gut, comprising:
(a) isolating each bacterium within a patient's stool sample by individually encapsulating the bacterium in an aqueous droplet surrounded by an oil-phase to obtain individually encapsulated bacterium;
(b) incubating each encapsulated bacterium under conditions appropriate for growth to obtain encapsulated bacterial strains;
(c) extracting DNA from each encapsulated bacterial strain at one or more time points during incubation;
(d) measuring:
(i) the quantity of total DNA extracted at each time point using quantitative Polymerase Chain Reaction (“qPCR”) via primers that target a variable region within a conserved gene sequence; and
(ii) the relative abundance at each time point of the encapsulated bacterial strain in the microflora through sequencing of the same variable region within a conserved gene sequence as in (i) using the same primers as in (i); and
(e) determining absolute growth based on the measurements obtained in (d) at each time point.

3. The method of claim 2, further comprising assessing the sensitivity of a bacterial strain from a mixed microbial community to an antibiotic drug, wherein (b) further comprises incubating each encapsulated bacterium both in the presence of an antibiotic drug and in the absence of an antibiotic drug, under conditions appropriate for growth to obtain a single encapsulated bacterial strain; and further comprising (f) measuring the growth of the bacterial strain in the presence of an antibiotic drug and growth in the absence of an antibiotic drug.

4. The method of claim 3, wherein the bacterial strain is sensitive to an antibiotic drug when the measurement in (f) demonstrates that the bacterial strain grown in the presence of an antibiotic drug exhibits less than 50% of the growth of the same bacterial strain grown in the absence of an antibiotic drug.

5. The method of claim 3, wherein the antibiotic drug comprises amoxicillin, amoxicillin clavulanate, ciprofloxacin, gentamicin, kanamycin or ampicillin.

6. The method of claim 2, further comprising assessing the ability of a gut bacterial strain to inactivate a xenobiotic, wherein (b) further comprises incubating each encapsulated bacterium both in the presence of a xenobiotic and in the absence of a xenobiotic, under conditions appropriate for growth to obtain a single encapsulated bacterial strain; and further comprising (f) measuring the growth of the bacterial strain in the presence of the xenobiotic and growth in the absence of the xenobiotic.

7. The method of claim 2, further comprising assessing the ability of a gut bacterial strain to degrade a prebiotic, wherein (b) further comprises incubating each encapsulated bacterium both in the presence of a prebiotic and in the absence of a prebiotic, under conditions appropriate for growth to obtain a single encapsulated bacterial strain; and further comprising (f) measuring the growth of the bacterial strain in the presence of the prebiotic and growth in the absence of the prebiotic.

8. The method of claim 1, wherein isolation of the bacterium via encapsulation is random.

9. The method of claim 1, wherein the encapsulated bacterium is incubated under anaerobic conditions.

10. The method of claim 1, wherein the aqueous droplet encapsulating the bacterium comprises nutrient-rich culture medium.

11. The method of claim 10, wherein the nutrient-rich culture medium comprises Brain Heart Infused (BHI) medium, Gifu Anaerobic Medium (GAM) or modified Gifu Anaerobic Medium (mGAM).

12. The method of claim 9, wherein the nutrient-rich culture media is modified to include a change in one or more of the following: oxygen (O₂) concentration, pH, or salt (NaCl) concentration.

13. The method of claim 1, wherein the aqueous droplet encapsulating the bacterium comprises defined medium.

14. The method of claim 13, wherein the defined medium comprises a carbon source.

15. The method of claim 14, wherein the carbon source is a dietary carbon source selected from the group consisting of: mannose, arabinose, fructose, glucose, xylan, lamanarin, pullalan, levan, rice-starch, arabinogalactan, inulin, fructooligosaccharide (FOS), galactose, glucuronic acid, pectin, galactomannan, guar gum, chitin, galacto-oligosaccarides, cellobiose, dextran, and beta-glucan.

16. The method of claim 13, wherein the defined medium comprises an amino acid.

17. The method of claim 1, wherein the aqueous droplets encapsulating the bacterium are incubated at 37° C.

18. The method of claim 1, wherein the aqueous droplets encapsulating the bacterium are incubated at 39° C.

19. The method of claim 1, wherein the primers comprise: GTGCCAGCMGCCGCGGTAA (SEQ ID NO:1) and GGACTACHVGGGTWTCTAAT (SEQ ID NO:2).

20. The method of claim 1, wherein the absolute growth is determined by multiplying the relative abundance sequencing data obtained for each time point by the quantitative qPCR data obtained for each time point, respectively.

21. The method of claim 1, wherein the absolute growth is measured by dividing the absolute number of cells at T24 by the absolute number of cells at T0.

22. The method of claim 6, wherein the xenobiotic comprises an ingested chemical that is not naturally produced by an organism.

23. The method of claim 6, wherein the xenobiotic comprises a therapeutic, such as a drug, a drug component, or a preservative.

24. A library of bacteria that display a targeted phenotype, wherein the bacteria are generated according to the method of claim 1.


SEQUENCE LISTING (2 sequences)

SEQ ID NO: 1 - DNA, 19 bp, Artificial Sequence
  16s V4 forward primer
gtgccagcmgccgcggtaa

SEQ ID NO: 2 - DNA, 20 bp, Artificial Sequence
  16s V4 rRNA reverse primer
ggactachvgggtwtctaat